(19) United States
(12) Patent Application Publication
(10) Pub. No.: US 2024/0247029 A1
(43) Pub. Date: Jul. 25, 2024

(54) INTERLEUKIN-1 RECEPTOR MODULATORS AND USES THEREOF

(71) Applicants: VALORISATION HSJ, LIMITED PARTNERSHIP, Québec (CA); VALORISATION-RECHERCHE, LIMITED PARTNERSHIP, Québec (CA)

(72) Inventors: Xin HOU, Saint-Laurent (CA); Azade GERANURIMI, Pierrefonds (CA); William D. LUBELL, Outremont (CA); Sylvain CHEMTOB, Côte-St-Luc (CA); Christiane QUINIOU, Montréal (CA); Colin CHENG, Montréal (CA)

(21) Appl. No.: 18/043,230

(22) PCT Filed: Aug. 27, 2021
(86) PCT No.: PCT/CA2021/051192

§ 371 Date: Feb. 27, 2023

Related U.S. Application Data
(60) Provisional application No. 63/071,492, filed on Aug. 28, 2020.

(51) Int. Cl. C07K 7/02; A61P 27/02; A61K 38/00

(52) U.S. Cl. CPC C07K 7/02; A61P 27/02; A61K 38/00

(57) ABSTRACT

The present application relates to a compound of formula (I): (I) or a pharmaceutically acceptable salt thereof, as well as to pharmaceutical compositions comprising same. The use of such compounds, pharmaceutically acceptable salts thereof or pharmaceutical compositions for inhibiting the activity of an IL-I receptor in a cell, or for treating IL-1 related diseases, disorders or conditions such as inflammatory diseases in a subject, is also described.

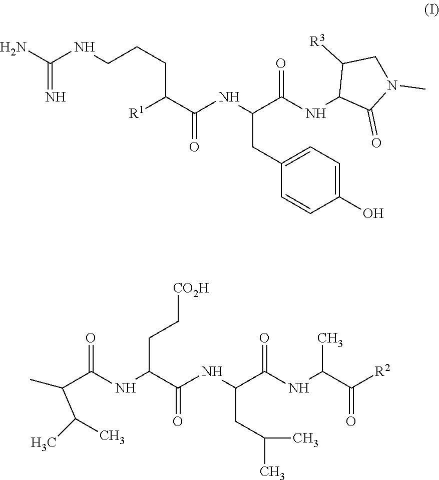

### CROSS REFERENCE TO RELATED APPLICATIONS

The present application claims the benefit of U.S. provisional patent application No. 63/071,492 filed on Aug. 28, 2020, which is incorporated herein by reference in its entirety.

### TECHNICAL FIELD

The present disclosure generally relates to the modulation of the Interleukin-1 receptor (IL-1R) and to the treatment of diseases or conditions associated with IL-1R activity such as inflammatory diseases or conditions.

### BACKGROUND ART

Inflammatory factor expression is induced primarily through signaling pathways triggered by interleukin-1 (IL-1β) (Gabay, Lamacchia, and Palmer 2010). This major pro-inflammatory cytokine stimulates various physiological effects leading ultimately to hyperthermia, hypotension, tissue destruction, and inflammation (Dinarello 1997). The activity of IL-1β is critical for inflammatory responses to treat damaged tissue and to ward off invading pathogens. Uncontrolled IL-1β activity is, however, a pathogenic characteristic of many chronic conditions.

In reproductive tissue during pregnancy, toll-like receptors (TLRs) can recognize and discriminate bacterial pathogen-associated molecular patterns (PAMPs) (Elovitz et al. 2003; Ilievski, Lu, and Hirsch 2007). The recognition of PAMPs from intra-amniotic infection (e.g., chorioamnionitis) leads to up-regulation and activation of TLRs (Kim et al. 2004) and release of pro-inflammatory IL-1β from immune cells. The latter plays a key role in the induction of both term and preterm labor (Romero et al. 1989). Engagement of TLRs also leads to the activation of nuclear factor kappa-light-chain-enhancer of activated B cells (NF-κB), a transcription factor that is involved in the expression of cytokines, such as IL-1β, chemokines, and defensin antimicrobial peptides (Choi, Scorpio, and Dumler 2004). Activation of NF-κB is important for maintaining immune vigilance against invading pathogens.

Regulation of TLR expression plays a key role in ischemic diseases of the retina, such as retinopathy of prematurity (ROP) (Xu and Wang 2016). Multiple cell types in the retina express TLRs, including glia, retinal pigment epithelium (RPE), photoreceptor, and endothelial cells. In the pathogenesis of retinal ischemic diseases, activation of TLRs initiates signal transduction, leading to production of pro-inflammatory cytokines, such as IL-1β (Xu and Wang 2016; Beaudry-Richard et al. 2018; Rivera et al. 2010; Rivera et al. 2017). In response to hypoxia, retinal microglia cells produce IL-1β and trigger an inflammatory cascade involving IL-6 and IL-8 (Tosato and Jones 1990). Endothelial cytotoxicity results from IL-1β-dependent retinal and sub-retinal injury in models of oxygen-induced retinopathy (OIR) (Tremblay et al. 2013; Zhou et al. 2016). Moreover, IL-1β has been linked to oxidative stress and associated with retinal microvascular degeneration mediated by the pro-apoptotic guidance cues of semaphorin 3A (Sema3A) (Rivera et al. 2013).

The IL-1 receptor (IL-1R) complex is composed of the IL-1 receptor I (IL-1R) and accessory protein (IL-1RAcP, occasionally referred to as IL1R3) subunits, which activate multiple signaling pathways upon binding to IL-1β (Krumm, Xiang, and Deng 2014). For example, the NF-κB protein complex is typically activated by IL-1β signaling which prompts cellular responses responsible for immuno-vigilance to counter bacterial and viral invasion. Other IL-1β-triggered signaling pathways involve kinases such as c-Jun N-terminal kinases (JNK) (Roy et al. 2008) and Rho-associated kinase-2 (ROCK2), (Amano, Nakayama, and Kaibuchi 2010) which regulate the inflammation cascade, including the synthesis of pro-inflammatory cytokines, and the maturation, migration, and activity of T cells.

Two natural inhibitors of IL-1β signaling are the intrinsic IL-1 receptor antagonist (IL-1Ra) and the decoy receptor, IL-1 receptor type II (IL-1RII) (Krumm, Xiang, and Deng 2014). IL-1Ra competes with IL-1 for the IL-1R binding site and prevents recruitment of IL-1RAcP. IL-1RII sequesters IL-1 but cannot form a signaling complex (Gabay, Lamacchia, and Palmer 2010). Current therapeutic strategies to counter pathological IL-1 signaling have been based on the mechanisms of these natural proteins. The three FDA-approved peptide-based therapies comprise: 1) the recombinant IL-1 receptor antagonist, Kineret® (anakinra), 2) the IL-1 Trap composed of a dimeric fusion of the ligand-binding domains of the extracellular portions of IL-1R1 and IL-1RAcP proteins, Rilonacept, and 3), a human monoclonal antibody targeting IL-1β, Canakinumab (Kaneko et al. 2019). These relatively large proteins have presented undesirable secondary effects in clinical settings including immunosuppression, which increases the risk for opportunistic infections, and pain at the site of injection (Opal et al. 1997; Roerink et al. 2017). Their failures in clinical trials may be due in part to drawbacks related to acting directly on the native orthosteric ligand and non-selectively interfering with all signals triggered by IL-1β (Opal et al. 1997; Roerink et al. 2017). Selective agents are desired to differentially target IL-1β signaling pathways leading to immune vigilance and inflammation. For example, allosteric ligands which bind remotely from the orthosteric ligand binding site of IL-1RI have been pursued to modulate IL-1β activity by inducing biased signaling. Such allosteric modulators may be smaller molecules exhibiting improved bioavailability, potential for oral administration, protease resistance and lower risks of toxicity.

The all D-amino acid heptapeptide rytvela (also named 101.10, 1, H-D-Arg-D-Tyr-D-Thr-D-Val-D-Glu-D-Leu-D-Ala-NH₂) was identified from a library of peptide sequences derived from the IL-1RAcP loop and juxtamembranous regions (Quiniou et al. 2008) Peptide 1 has exhibited potent, selective, and reversible non-competitive inhibition of IL-1β activity. For example, peptide 1 blocked IL-1β-induced human thymocyte cell proliferation in vitro (Jamieson et al. 2009) and demonstrated robust in vivo effects in models of hyperthermia and inflammatory bowel disease (Quiniou et al. 2008).

There is thus a need for the development of novel selective inhibitors of IL-1β signaling to treat inflammatory diseases.

The present description refers to a number of documents, the content of which is herein incorporated by reference in their entirety.

### SUMMARY

The present disclosure provides the following items 1 to 51:
1. A compound of formula (I):

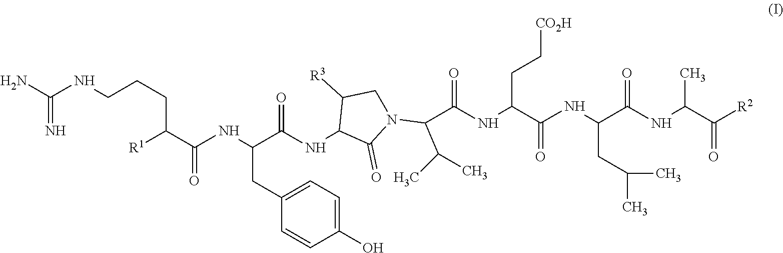

R¹ is H or NR⁴R⁵, wherein R⁴ and R⁵ are each independently H or R^{A}—CO—, wherein RA is a hydrophobic moiety or an aryl group;
R² is H, OH, C₁₋₆ alkyl, O-C₁₋₆ alkyl or NR⁶R⁷, wherein R⁶ and R⁷ are each independently H, OH, or C₁₋₆ alkyl;
R³ is S-C═N, NR⁶R⁷, optionally substituted phenyl; optionally substituted 3- to 7-membered saturated or partially unsaturated carbocyclic ring; optionally substituted 5- or 6-membered monocyclic heteroaryl ring having 1-3 heteroatoms independently selected from nitrogen, oxygen, and sulfur; or optionally substituted 4- to 7-membered saturated or partially unsaturated heterocyclic ring having 1-3 heteroatoms independently selected from nitrogen, oxygen, and sulfur;
   or a pharmaceutically acceptable salt thereof.
2. The compound or pharmaceutically acceptable salt thereof of item 1, wherein R⁴ and/or R⁵ are H.
3. The compound or pharmaceutically acceptable salt thereof of item 1 or 2, wherein R² is NR⁶R⁷.
4. The compound or pharmaceutically acceptable salt thereof of item 3, wherein R⁶ and/or R⁷ are H.
5. The compound or pharmaceutically acceptable salt thereof of any one of items 1 to 4, wherein R³ is S—C═N.
6. The compound or pharmaceutically acceptable salt thereof of any one of items 1 to 4, wherein R³ is NR⁶R⁷.
7. The compound or pharmaceutically acceptable salt thereof of item 6, wherein R⁶ and/or R⁷ are H.
8. The compound or pharmaceutically acceptable salt thereof of any one of items 1 to 4, wherein R³ is optionally substituted 5- or 6-membered monocyclic heteroaryl ring having 1-3 heteroatoms independently selected from nitrogen, oxygen, and sulfur.
9. The compound or pharmaceutically acceptable salt thereof of item 8, wherein R³ is optionally substituted 5-membered monocyclic heteroaryl ring having 1-3 nitrogen atoms, preferably R³ comprises a triazole ring.
10. The compound or pharmaceutically acceptable salt thereof of item 9, wherein R³ is

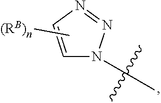

wherein
each R^{B} is independently hydroxy, halogen atom, alkyl, haloalkyl, alkoxy, cyano, amino, alkylamino, nitro, —N(haloalkyl)₂, aryl, heteroaryl, arylalkyl, arylalkyloxy, or —C(O)aryl; the alkyl, aryl, arylalkyl, heteroaryl, and alkylaryl in R^{B} being optionally substituted with one or more: hydroxy, halogen atom, alkyl, haloalkyl, alkoxy, cyano, amino, alkylamino, nitro, —N(haloalkyl)₂, aryl, arylalkyl, arylalkyloxy, or —C(O)aryl;
n is 0, 1 or 2.
11. The compound or pharmaceutically acceptable salt thereof of item 10, wherein R³ is

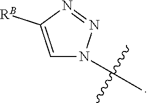

12. The compound or pharmaceutically acceptable salt thereof of item 10 or 11, wherein R³ is:

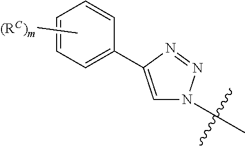

wherein
each R^{C} is independently hydroxy, halogen atom, alkyl, haloalkyl, alkoxy, cyano, amino, alkylamino, nitro, or -N(haloalkyl)₂; and
13. The compound or pharmaceutically acceptable salt thereof of item 12, wherein m is 0.
14. The compound or pharmaceutically acceptable salt thereof of any one of items 1 to 13, which is a compound of formula la:

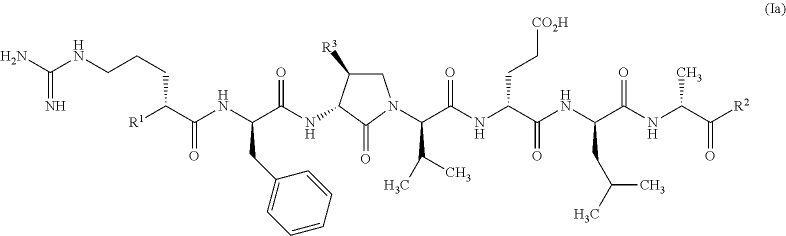

or a pharmaceutically acceptable salt thereof.
15. The compound or pharmaceutically acceptable salt thereof of item 1, which is a compound of one of the following structures:

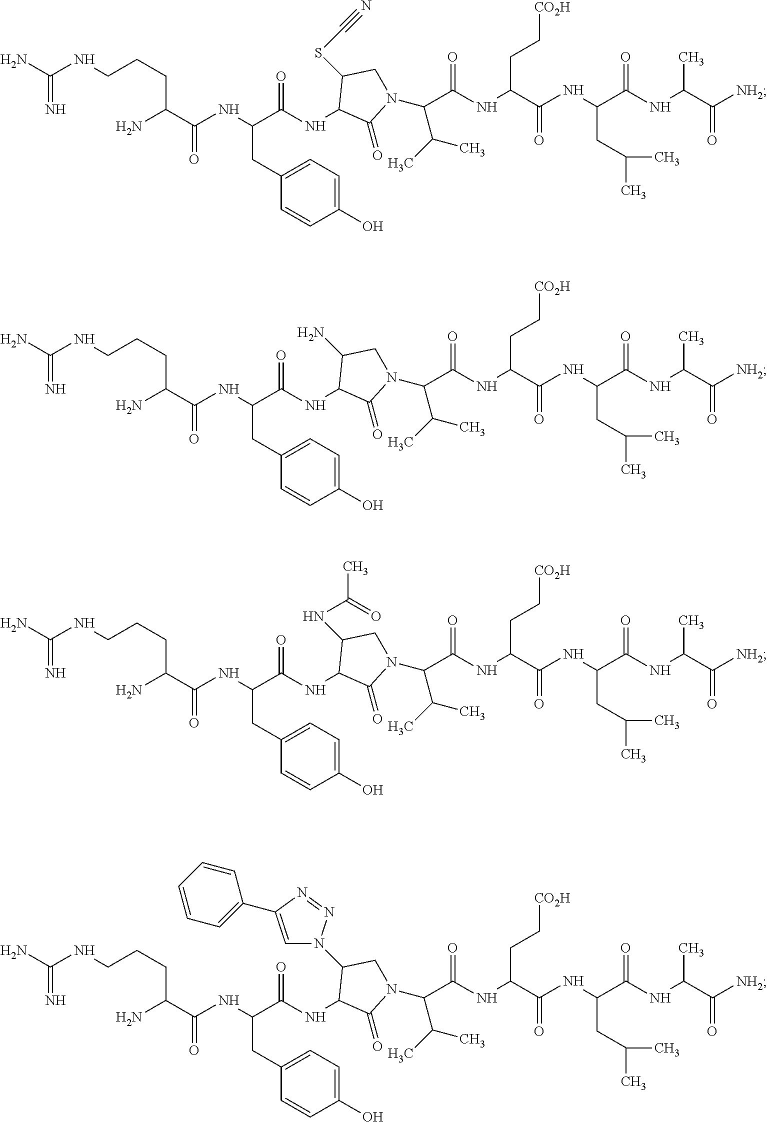 [SMILES: CC(C)CC(NC(=O)C(CCC(=O)O)NC(=O)C(C(C)C)N1CC(SC#N)C(NC(=O)C(Cc2ccc(O)cc2)NC(=O)C(N)CCCNC(=N)N)C1=O)C(=O)NC(C)C(N)=O ; CC(C)CC(NC(=O)C(CCC(=O)O)NC(=O)C(C(C)C)N1CC(N)C(NC(=O)C(Cc2ccc(O)cc2)NC(=O)C(N)CCCNC(=N)N)C1=O)C(=O)NC(C)C(N)=O ; CC(=O)NC1CN(C(C(=O)NC(CCC(=O)O)C(=O)NC(CC(C)C)C(=O)NC(C)C(N)=O)C(C)C)C(=O)C1NC(=O)C(Cc1ccc(O)cc1)NC(=O)C(N)CCCNC(=N)N ; CC(C)CC(NC(=O)C(CCC(=O)O)NC(=O)C(C(C)C)N1CC(n2cc(-c3ccccc3)nn2)C(NC(=O)C(Cc2ccc(O)cc2)NC(=O)C(N)CCCNC(=N)N)C1=O)C(=O)NC(C)C(N)=O]

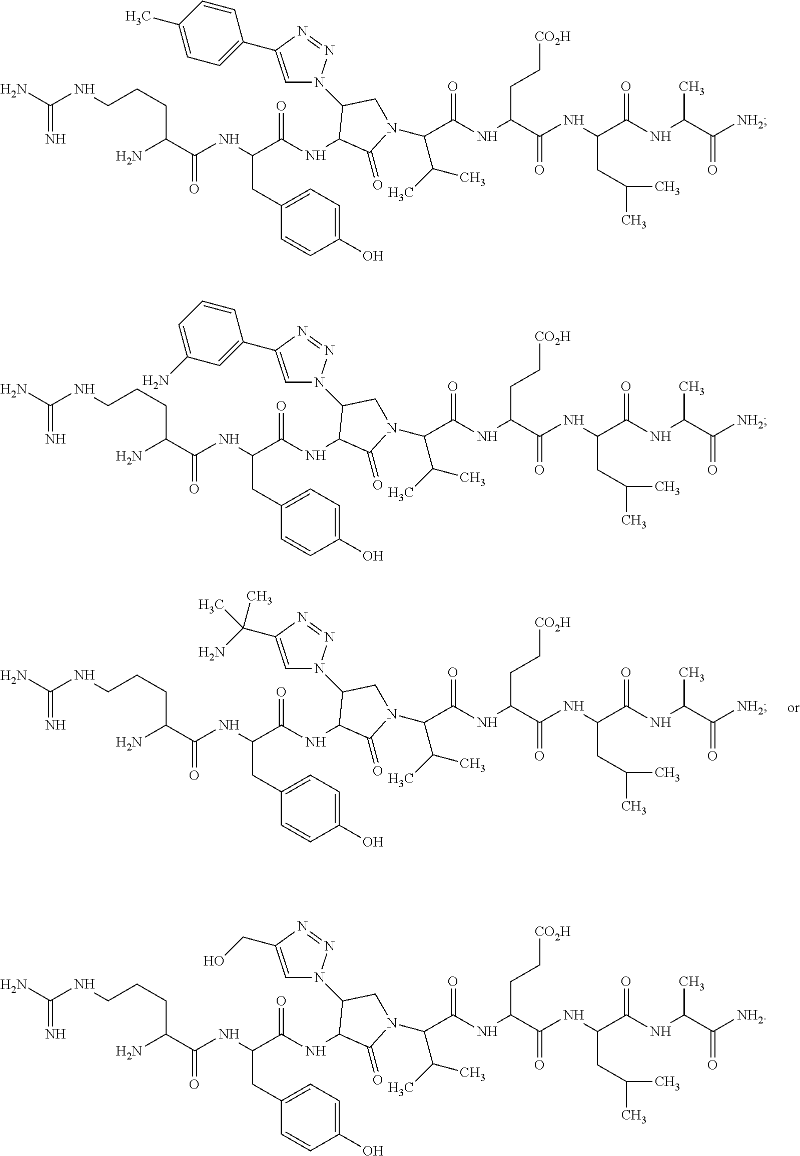

or a pharmaceutically acceptable salt thereof.
16. The compound or pharmaceutically acceptable salt thereof of item 15, which is a compound of one of the following structures:

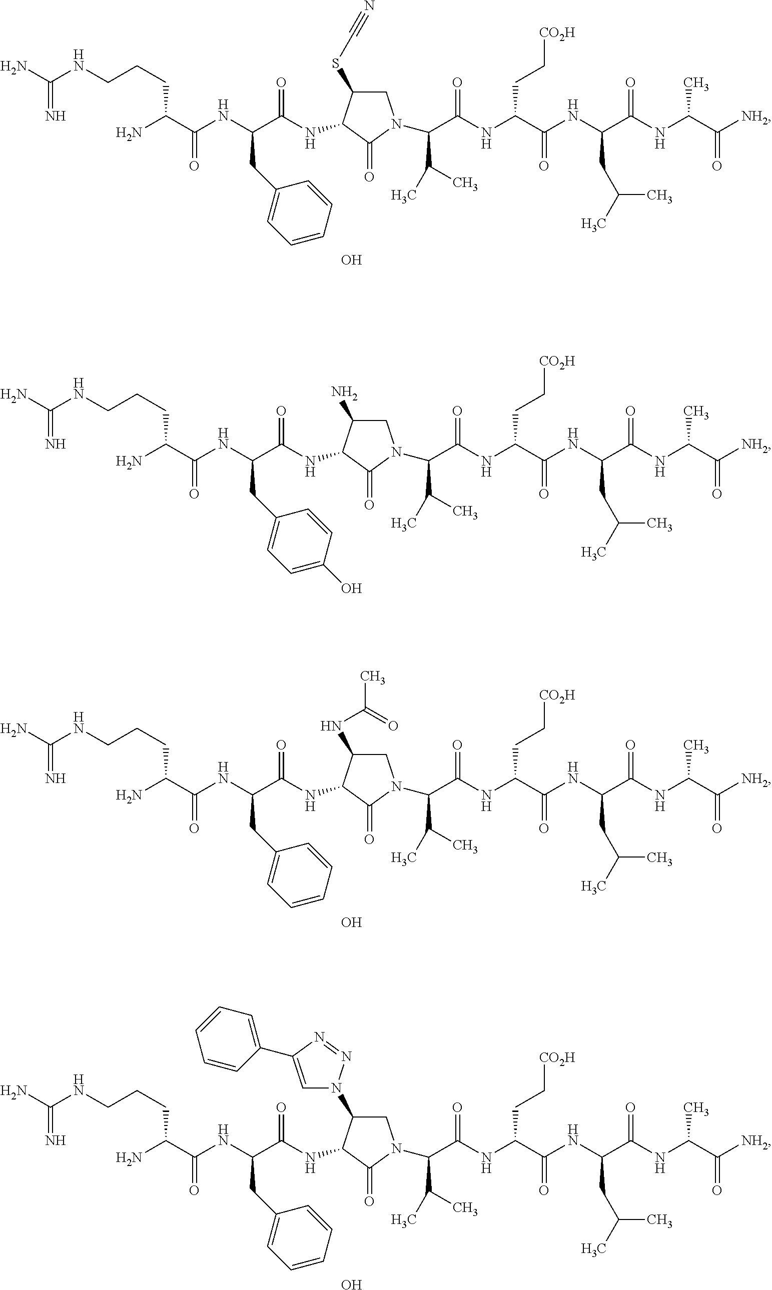

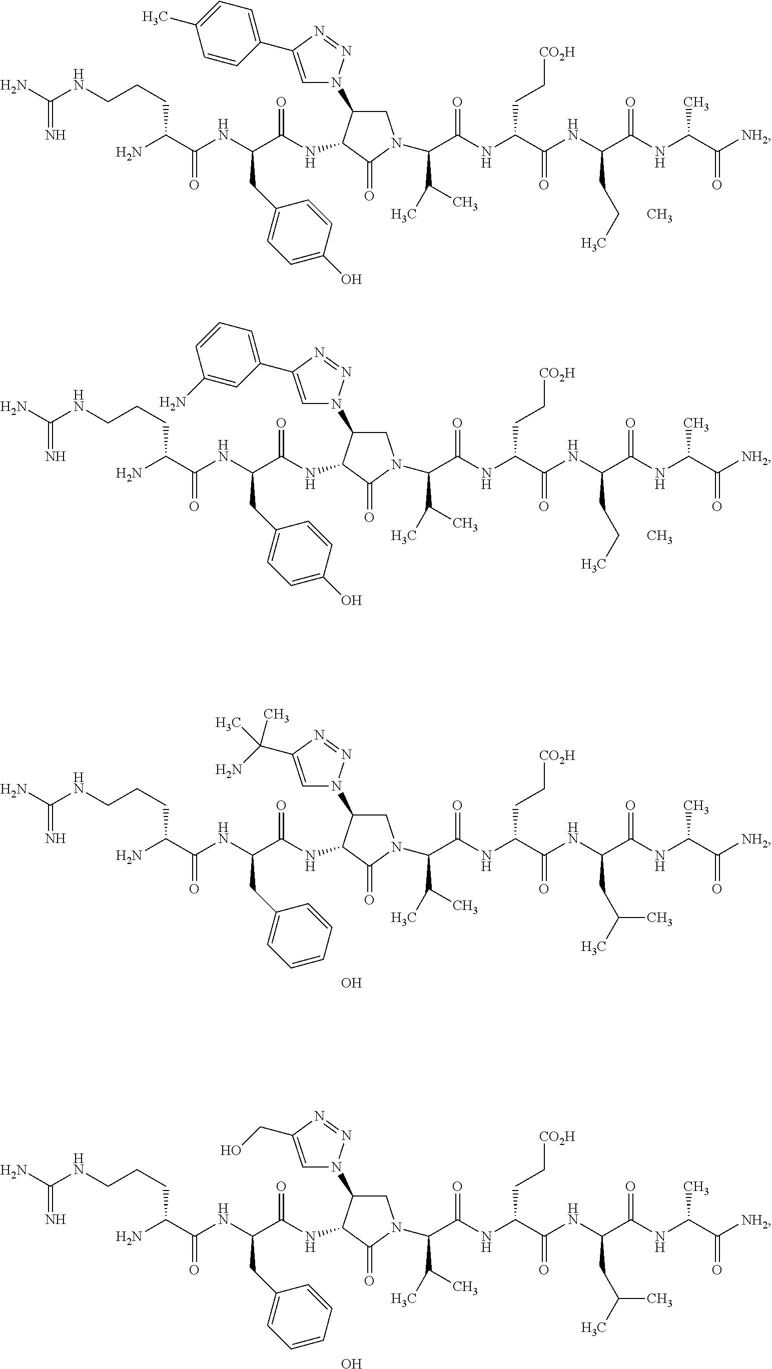

or a pharmaceutically acceptable salt thereof.
17. The compound or pharmaceutically acceptable salt thereof of item 16, which is

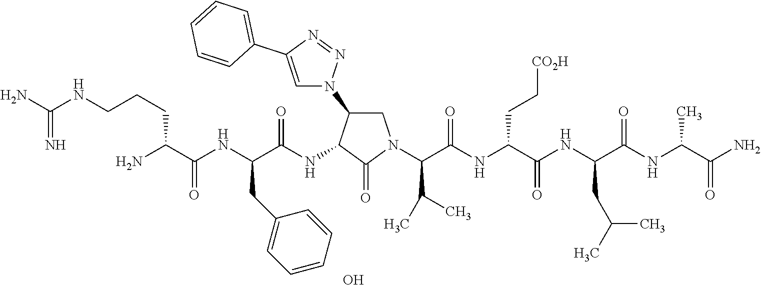

or a pharmaceutically acceptable salt thereof.
18. A pharmaceutical composition comprising the compound or pharmaceutically acceptable salt thereof of any one of items 1 to 17 and a pharmaceutically acceptable excipient.
19. A method of inhibiting the activity of an IL-1 receptor in a cell, the method comprising contacting the cell with the compound or pharmaceutically acceptable salt thereof of any one of items 1 to 17 or the pharmaceutical composition of item 18.
20. Use of the compound or pharmaceutically acceptable salt thereof of any one of items 1 to 17 or the pharmaceutical composition of item 18 for inhibiting the activity of an IL-1 receptor in a cell.
21. Use of the compound or pharmaceutically acceptable salt thereof of any one of items 1 to 17 or the pharmaceutical composition of item 18 for the manufacture of a medicament for inhibiting the activity of an IL-1 receptor in a cell.
22. The compound or pharmaceutically acceptable salt thereof of any one of items 1 to 17 or the pharmaceutical composition of item 18 for use in inhibiting the activity of an IL-1 receptor in a cell.
23. A method of treating an IL-1 related disease, disorder or condition, the method comprising administering to a subject in need of treatment the compound or pharmaceutically acceptable salt thereof of any one of items 1 to 17 or the pharmaceutical composition of item 18.
24. The method of item 23, wherein the IL-1 related disease, disorder or condition is an inflammatory disease, disorder or condition.
25. The method of item 24, wherein the inflammatory disease, disorder or condition is inflammatory joint disease, inflammatory bowel disease, psoriasis, encephalitis, glomerulonephritis, respiratory distress syndrome, Reiter's syndrome, systemic lupus erythematosus, scleroderma, stroke, periventricular leucopenia, meningitis, multiple sclerosis, acute disseminated encephalomyelitis (ADEM), idiopathic inflammatory demyelinating disease, transverse myelitis, Devic's disease, progressive multifocal leukoencephalopathy (PML), Guillain-Barre syndrome, age-related macular degeneration (AMD), retinopathy, chronic inflammatory demyelinating polyneuropathy, anti-MAG neuropathy, sepsis, septic shock, pancreatitis, trauma-induced shock, asthma, allergic rhinitis, cystic fibrosis, acute bronchitis, chronic bronchitis, acute bronchiolitis, chronic bronchiolitis, gout, placental, fetal and/or uterine inflammation, inflammatory condition resulting from an injury, or inflammatory condition resulting from an infection.
26. The method of item 25, wherein the inflammatory joint disease is rheumatoid arthritis or osteoarthritis.
27. The method of item 25, wherein the inflammatory bowel disease is Crohn's disease or ulcerative colitis.
28. The method of item 25, wherein the retinopathy is diabetic retinopathy (DR) or retinopathy of prematurity (ROP).
29. The method of item 25, wherein the placental, fetal and/or uterine inflammation causes preterm labor.
30. The method of item 25, wherein the inflammatory condition resulting from an infection is sepsis or acute respiratory distress syndrome (ARDS).
31. Use of the compound or pharmaceutically acceptable salt thereof of any one of items 1 to 15 or the pharmaceutical composition of item 16 for treating an IL-1 related disease, disorder or condition in a subject.
32. Use of the compound or pharmaceutically acceptable salt thereof of any one of items 1 to 15 or the pharmaceutical composition of item 16 for the manufacture of a medicament for treating an IL-1 related disease, disorder or condition in a subject.
33. The use of item 31 or 32, wherein the IL-1 related disease, disorder or condition is an inflammatory disease, disorder or condition.
34. The use of item 33, wherein the inflammatory disease, disorder or condition is inflammatory joint inflammatory bowel disease, psoriasis, encephalitis, glomerulonephritis, respiratory distress syndrome, Reiter's syndrome, systemic lupus erythematosus, scleroderma, stroke, periventricular leucopenia, meningitis, multiple sclerosis, acute disseminated encephalomyelitis (ADEM), idiopathic inflammatory demyelinating disease, transverse myelitis, Devic's disease, progressive multifocal leukoencephalopathy (PML), Guillain-Barre syndrome, age-related macular degeneration (AMD), retinopathy, chronic inflammatory demyelinating polyneuropathy, anti-MAG neuropathy, sepsis, septic shock, pancreatitis, trauma-induced shock, asthma, allergic rhinitis, cystic fibrosis, acute bronchitis, chronic bronchitis, acute bronchiolitis, chronic bronchiolitis, gout, placental, fetal and/or uterine inflammation, inflammatory condition resulting from an injury, or inflammatory condition resulting from an infection.
35. The use of item 34, wherein the inflammatory joint disease is rheumatoid arthritis or osteoarthritis.
36. The use of item 34, wherein the inflammatory bowel disease is Crohn's disease or ulcerative colitis.
37. The use of item 34, wherein the retinopathy is diabetic retinopathy (DR) or retinopathy of prematurity (ROP).
38. The use of item 34, wherein the placental, fetal and/or uterine inflammation causes preterm labor.
39. The use of item 34, wherein the inflammatory condition resulting from an infection is sepsis or acute respiratory distress syndrome (ARDS).
40. The compound or pharmaceutically acceptable salt thereof of any one of items 1 to 17 or the pharmaceutical composition of item 18 for use in treating an IL-1 related disease, disorder or condition in a subject.
41. The compound, pharmaceutically acceptable salt thereof or pharmaceutical composition for use according to item 40, wherein the IL-1 related disease, disorder or condition is an inflammatory disease, disorder or condition.
42. The compound, pharmaceutically acceptable salt thereof or pharmaceutical composition for use according to item 41, wherein the inflammatory disease, disorder or condition is inflammatory joint disease, inflammatory bowel disease, psoriasis, encephalitis, glomerulonephritis, respiratory distress syndrome, Reiter's syndrome, systemic lupus erythematosus, scleroderma, stroke, periventricular leucopenia, meningitis, multiple sclerosis, acute disseminated encephalomyelitis (ADEM), idiopathic inflammatory demyelinating disease, transverse myelitis, Devic's disease, progressive multifocal leukoencephalopathy (PML), Guillain-Barre syndrome, age-related macular degeneration (AMD), retinopathy, chronic inflammatory demyelinating polyneuropathy, anti-MAG neuropathy, sepsis, septic shock, pancreatitis, trauma-induced shock, asthma, allergic rhinitis, cystic fibrosis, acute bronchitis, chronic bronchitis, acute bronchiolitis, chronic bronchiolitis, gout, placental, fetal and/or uterine inflammation, inflammatory condition resulting from an injury, or inflammatory condition resulting from an infection.
43. The compound, pharmaceutically acceptable salt thereof or pharmaceutical composition for use according to item 42, wherein the inflammatory joint disease is rheumatoid arthritis or osteoarthritis.
44. The compound, pharmaceutically acceptable salt thereof or pharmaceutical composition for use according to item 42, wherein the inflammatory bowel disease is Crohn's disease or ulcerative colitis.
45. The compound, pharmaceutically acceptable salt thereof or pharmaceutical composition for use according to item 42, wherein the retinopathy is diabetic retinopathy (DR) or retinopathy of prematurity (ROP).
46. The compound, pharmaceutically acceptable salt thereof or pharmaceutical composition for use according to item 42, wherein the placental, fetal and/or uterine inflammation causes preterm labor.
47. The compound, pharmaceutically acceptable salt thereof or pharmaceutical composition for use according to item 42, wherein the inflammatory condition resulting from an infection is sepsis or acute respiratory distress syndrome (ARDS).
48. A method for preventing preterm birth (PTB) and/or for preventing or reducing the risk of perinatal or neonatal morbidity and mortality caused by antenatal fetal inflammation, comprising administering to an expectant mother in need thereof an effective amount of the compound or pharmaceutically acceptable salt thereof of any one of items 1 to 17 or the pharmaceutical composition of item 18.
49. Use of the compound or pharmaceutically acceptable salt thereof of any one of items 1 to 17 or the pharmaceutical composition of item 18 for preventing preterm birth (PTB) and/or for preventing or reducing the risk of perinatal or neonatal morbidity and mortality caused by antenatal fetal inflammation.
50. Use of the compound or pharmaceutically acceptable salt thereof of any one of items 1 to 17 or the pharmaceutical composition of item 18 for the manufacture of a medicament for preventing preterm birth (PTB) and/or for preventing or reducing the risk of perinatal or neonatal morbidity and mortality caused by antenatal fetal inflammation.
51. The compound or pharmaceutically acceptable salt thereof of any one of items 1 to 17 or the pharmaceutical composition of item 18 for use in preventing preterm birth (PTB) and/or for preventing or reducing the risk of perinatal or neonatal morbidity and mortality caused by antenatal fetal inflammation.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the structure of B-Substituted-Agl analogs 2 and 3.

FIG. 2 shows the molar ellipticity circular dichroism spectra of compounds 2b-d, 2j, and 2;

FIG. 3 is a graph showing the effects of peptides 1 and 2 on IL-1β-induced NF-κB signaling in HEK-Blue cells as quantified in the QUANTI-blue assay. After pre-incubation with peptide or vehicle, HEK-Blue cells were stimulated with IL-1β for 24 h, and secreted alkaline phosphatase activity was spectroscopically detected as a reporter product from the transcription of the NF-κB gene. Data shown represents the average of 2 experiments (each with n=4 per treatment group): ****p<0.0001 compared to group treated only with IL-1β.

FIGS. 4A and 4B are graphs showing the results of qPCR analysis of IL-1β (FIG. 4A) and COX-2 (FIG. 4B) gene expression within lysates from RAW 264.7 mouse macrophage cells after exposure to peptides 1 and 2 (10-6 M) or Kineret (1 mg/mL) followed by IL-13 incubation overnight. Results represent an average of 3 independent experiments (each with n=4 per treatment group) and are expressed as a fold-change of the non-stimulated control: *p<0.05, **p<0.01 compared to group treated only with IL-1β, and 18S rRNA as internal control. Treatment groups that are not labeled with asterisks are statistically non-significant compared to group treated only with IL-1β.;

FIGS. 5A-C are graphs showing the effects of peptides 1 and 2 on IL-1β-induced phosphorylation of p38 (FIG. 5A), JNK (FIG. 5B), and ROCK2 (FIG. 5C). Graphical representations of band density analysis of Western Blots are shown as fold activation compared to control. RAW Blue cells were pretreated with peptides 1 and 2 (10⁻⁶ M), Kineret (1 mg/mL), or vehicle for 30 min and then stimulated with IL-1β for 15 min. Results shown are the average of 3 independent experiments: *p<0.05, ** p<0.01, ***p<0.001 compared to group treated only with IL-1β. Treatment groups that are not labeled with asterisks are statistically non-significant compared to group treated only with IL-1β.

FIG. 6 is a graph showing the effects of peptides 1 and 2 on prevention of PTB. In brief, pregnant dams on day 16.5 of gestation (G16.5) were subcutaneously pretreated with peptides 1 and 2 (2 mg/kg/day subcutaneous injections) or vehicle, followed by LPS (10 μg intraperitoneal injection), and observed for delivery of pups. A dam was considered as delivering preterm if at least one pup was delivered before G18.5. n=4-5 dams per treatment group.

FIG. 7A is a representative retinal flatmounts stained with FITC-conjugated Bandeiraea simplicifolia lectin at 10X magnification following treatment with peptides 1 and 2. Lines indicate the central area of vaso-obliteration extending from the optic nerve in the center of the retina.

FIG. 7B is a graph showing the quantification of area of vaso-obliteration performed using ImageJ, expressed as a percentage of the total retinal area: n=5-7 of peptide 2, n=10-12 for vehicle and peptide 1; Veh=vehicle; ****p<0.0001 relative to the vehicle group, n.s. p>0.05 relative to vehicle group and not statistically significant.

FIG. 8A are representative confocal images of retinal microglia at 30× magnification following treatment with peptides 1 and 2: scale bar 100 μm.

FIG. 8B is a graph showing the epifluorescence microscopy images at 20× magnification of retinal microglial density quantified using ImageJ: 4 images per retina were taken at a distance halfway between the optic nerve and the edge of the retina; n=5-7 for peptides 1 and 2; n=10-12 for normoxia and vehicle; Norm =normoxia, Veh =vehicle; ****p<0.0001 relative to the vehicle group, n.s. p>0.05 relative to vehicle group and not statistically significant.

FIG. 9 depicts a summary of the results obtained with the peptides in the different models tested in the studies described herein.

### DETAILED DISCLOSURE

The use of the terms “a” and “an” and “the” and similar referents in the context of describing the invention are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”, etc.) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

Any and all combinations and subcombinations of the embodiments and features disclosed herein are encompassed by the present invention.

Herein, the terms “alkyl”, “alkylene”, “alkenyl”, “alkenylene”, “alkynyl”, “alkynylene” and their derivatives (such as alkoxy, alkyleneoxy, etc.) have their ordinary meaning in the art. For more certainty, herein:

| Term | Definition |
|---|---|
| alkyl | monovalent saturated aliphatic hydrocarbon radical of |
| | general formula —CₙH₂ₙ₊₁ |
| alkenyl | monovalent aliphatic hydrocarbon radical similar to an |
| | alkyl, but comprising at least one double bond |
| alkynyl | monovalent aliphatic hydrocarbon radical similar to an |
| | alkyl, but comprising at least one triple bond |
| alkenynyl | monovalent aliphatic hydrocarbon radical similar to an |
| | alkyl, but comprising at least one double bond |
| | and at least one triple bond |
| alkylene | bivalent saturated aliphatic hydrocarbon radical of |
| | general formula —CₙH₂ₙ— (also called alkanediyl) |
| alkenylene | bivalent aliphatic hydrocarbon radical similar to an |
| | alkylene, but comprising at least one double bond |
| alkynylene | bivalent aliphatic hydrocarbon radical similar to an |
| | alkylene, but comprising at least one triple bond |
| alkenynylene | bivalent aliphatic hydrocarbon radical similar to an |
| | alkylene, but comprising at least one double bond |
| | and at least one triple bond |
| alkyloxy or alkoxy | monovalent radical of formula —O-alkyl |
| alkyleneoxy | binovalent radical of formula —O-alkylene- |
| alkenyloxy | monovalent radical of formula —O-alkenyl |
| alkenyleneoxy | binovalent radical of formula —O-alkenylene- |
| alkynyloxy | monovalent radical of formula —O-alkynyl |
| alkynyleneoxy | binovalent radical of formula —O-alkynylene |
| haloalkyl | alkyl radical substituted with one or more halogen atom |
| | (the halogen atoms being the same or different, preferably |
| | being the same), up to and including perhaloalkyls in |
| | which the alkyl is completely substituted with halogen |
| | atoms |

It is to be noted that, unless otherwise specified, the hydrocarbon chains of these groups can be linear or branched. Further, unless otherwise specified, these groups can contain between 1 and 18 carbon atoms, more specifically between 1 and 12 carbon atoms, between 1 and 6 carbon atoms, between 1 and 3 carbon atoms, or contain 1 or 2 carbon atoms.

Herein, the terms “cycloalkyl”, “aryl”, “heterocycloalkyl”, “heteroaryl” have their ordinary meaning in the art. For more certainty, herein

| Term | Definition |
|---|---|
| cycloalkyl | monovalent saturated aliphatic hydrocarbon radical of |
| | general formula CₙH₂ₙ₋₁, wherein the carbon atoms are |
| | arranged in a ring (also called cycle). |
| heterocycloalkyl | cycloalkyl wherein at least one of the carbon atoms is |
| | replaced by a heteroatom, such as nitrogen or oxygen. |
| cycloalkylalkyl | monovalent radical of formula -alkyl, wherein the alkyl |
| | is substituted, preferably end-substituted, with |
| | cycloalkyl |
| aryl | monovalent aromatic hydrocarbon radical presenting a |
| | delocalized conjugated IT system, most commonly an |
| | arrangement of alternating double and single bonds, |
| | between carbon atoms arranged in one or more rings, |
| | wherein the rings can be fused (i.e. share two ring |
| | atoms), for example: |
| | naphthalene: |
| | 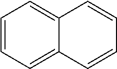 |
| | or linked together through a covalent bond, for |
| | example: |
| | biphenyl: |
| | 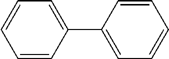 [SMILES: c1ccc(-c2ccccc2)cc1] |
| | or linked together through a radical that allow |
| | continuation of the delocalized conjugated TT system |
| | between the rings (e.g. —C(═O)—, —NRR—), |
| | for example: |
| | benzophenone: |
| | 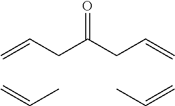 |
| aryloxy | monovalent radical of formula —O-aryl |
| arylalkyl | monovalent radical of formula -alkyl, wherein the alkyl |
| | is substituted, preferably end-substituted with aryl |
| arylalkylene | bivalent radical of formula -alkylene-, wherein the |
| | alkylene is substituted, with aryl |
| heteroaryl | aryl wherein at least one of the carbon atoms is re- |
| | placed by a heteroatom, such as nitrogen or oxygen. |
| heteroarylalkyl | monovalent radical of formula -alkyl, wherein the alkyl |
| | is substituted, preferably end-substituted, with |
| | heteroaryl |

It is to be noted that, unless otherwise specified, the ring of the above groups can comprise between 4 and 8, preferably 5 or 6 ring atoms. Further, unless otherwise specified, these groups can contain a total of between 5 and 12 carbon atoms, preferably between 5 and 6 carbon atoms disposed in a single ring, or between 9 to 12 carbon atoms disposed into two rings. A preferred aryl groups is phenyl.

The terms “halo” and “halogen” as used herein refer to an atom selected from fluorine (fluoro, —F), chlorine (chloro, —CI), bromine (bromo, —Br), and iodine (iodo, —I).

In the studies described herein, the present inventors have developed novel selective, and reversible non-competitive inhibitors of IL-1β activity.

Accordingly, in a first aspect, the present disclosure provides a compound of formula (I):

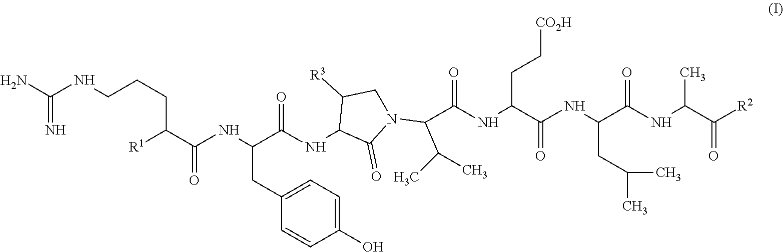

wherein:
R¹ is H or NR⁴R⁵, wherein R⁴ and R⁵ are each independently H or RA-CO-, wherein RA is a hydrophobic moiety or an aryl group;
R² is H, OH, C1-6 alkyl, O-C1-6 alkyl or NR⁶R⁷, wherein R⁶ and R⁷ are each independently H, OH, or C1-6 alkyl;
R³ is S—C═N, NR⁶R⁷, optionally substituted phenyl; optionally substituted 3- to 7-membered saturated or partially unsaturated carbocyclic ring; optionally substituted 5- or 6-membered monocyclic heteroaryl ring having 1-3 heteroatoms independently selected from nitrogen, oxygen, and sulfur; or optionally substituted 4- to 7-membered saturated or partially unsaturated heterocyclic ring having 1-3 heteroatoms independently selected from nitrogen, oxygen, and sulfur;
or a pharmaceutically acceptable salt thereof.
R¹ corresponds to the amino-terminal end of the peptide compound and may be the native NH₂ group of the peptide, or may comprise an amino-terminal modifying group. The term “amino-terminal modifying group” refers to a moiety commonly used in the art of peptide chemistry to replace or modify the native NH₂ terminal group of the peptide, for example to increase its stability and/or susceptibility to protease digestion. In an embodiment, R⁴ and/or R⁵ is a straight chained or branched alkyl group of one to eight carbons, or an acyl group (R^{A}—CO—), wherein RA is a hydrophobic moiety (e.g., alkyl, such as methyl, ethyl, propyl, butanyl, iso-propyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated); in a further embodiment, a saturated C₁-C₆ acyl group (linear or branched) or an unsaturated C₃-C₆ acyl group (linear or branched), for example an acetyl group (CH₃—CO—, Ac). In an embodiment, R⁴ and/or R⁵ represent a hydrogen atom (i.e., the cyclic peptide has a native NH₂ terminal group) or an acyl group (linear or branched), such as a C₁-C₆ acyl group, preferably an acetyl group (CH₃—CO—, Ac). In more preferred embodiments, R⁴ and R⁵ represent a hydrogen atom.
R² corresponds to the carboxy-terminal end of the peptide compound and may be hydroxyl to form the native COOH group of the peptide (with the adjacent carboxyl group), or may comprise a carboxy-terminal modifying group. The term “carboxy-terminal modifying group” refers to a moiety commonly used in the art of peptide chemistry to replace or modify the native CO₂H terminal group of the peptide, for example to increase its stability and/or susceptibility to protease digestion. In embodiment, the carboxy-terminal modifying group is:
   a hydroxylamine group (NHOH) attached to the carboxyl group (—C(═O)—NHOH), or
   an amine attached to the carboxyl group (—C(═O)—NR²⁰R²¹), the amine being a primary, secondary or tertiary amine, and preferably the amine is an aliphatic amine preferably of one to ten carbons, such as methyl amine, iso-butylamine, iso-valerylamine or cyclohexylamine, an aromatic amine or an arylalkyl amine, such as aniline, napthylamine, benzylamine, cinnamylamine, or phenylethylamine, a preferred amine being —NH₂,

In an embodiment, R² represents OH (i.e., the cyclic peptide has a native CO2H terminal group). In another embodiment, R² is a carboxy-terminal modifying group, preferably NH₂.

In an embodiment, R³ is S-C═N.

In an embodiment, R³ is NR⁶R⁷, wherein R⁶ and R⁷ are each independently H, OH, or C₁-6 alkyl. In a further embodiment, at least one of R⁶ and R⁷ is H. In a further embodiment, R⁶ and R⁷ are H.

In an embodiment, R³ is optionally substituted 5- or 6-membered monocyclic heteroaryl ring having 1-3 heteroatoms independently selected from nitrogen, oxygen, and sulfur. In an embodiment, R³ is a substituted 5- or 6-membered monocyclic heteroaryl ring having 1-3 heteroatoms independently selected from nitrogen, oxygen, and sulfur. In an embodiment, R³ is a substituted 5-membered monocyclic heteroaryl ring, preferably a substituted 5-membered monocyclic heteroaryl ring having 1-3 nitrogen atoms.

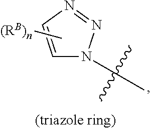

In an embodiment, R³ is
wherein each RB is independently hydroxy, halogen atom, alkyl, haloalkyl, alkoxy, cyano, amino, alkylamino, nitro, —N(haloalkyl)₂, aryl, heteroaryl, arylalkyl, heteroarylalkyl, arylalkyloxy, or —C(O)aryl;
the alkyl, aryl, arylalkyl, heteroaryl, and heteroarylalkyl in RB being optionally substituted with one or more: hydroxy, halogen atom, alkyl, haloalkyl, alkoxy, cyano, amino, alkylamino, nitro, —N(haloalkyl)₂, aryl, heteroarylalkyl, arylalkyl, arylalkyloxy, or —C(O)aryl;
n is 0, 1 or 2.

In an embodiment, R³ is

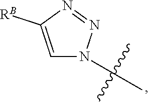

wherein R^{B} and n are as defined above.

In an embodiment, R^{B} is aryl, preferably phenyl.

In an embodiment, R³ is

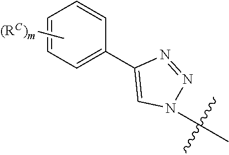

wherein each R^{C} is independently hydroxy, halogen atom, alkyl, haloalkyl, alkoxy, cyano, amino, alkylamino, nitro, or —N(haloalkyl)₂;
m is 0, 1, 2 or 3, preferably 0 or 1, more preferably 0.

The compounds of formula I described herein have at least one asymmetric carbon atoms and can therefore exist in the form of optically pure enantiomers, as racemates and as mixture thereof. Some of the compounds have at least two asymmetric carbon atoms and can therefore exist in the form of pure distereoisomers and as mixtures thereof.

The synthesis of optically active forms may be carried out by standard techniques of organic chemistry well known in the art, for example by resolution of the racemic form by recrystallisation techniques, by chiral synthesis, by enzymatic resolution, by biotransformation or by chromatographic separation. More specifically, diastereomeric mixtures can be separated into their individual diastereomers on the basis of their physical chemical differences by methods well known to those skilled in the art, such as, for example, by chromatography and/or fractional crystallization. Enantiomers can be separated, for example, by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers.

As used herein, the term “optical isomers” of compounds of the present disclosure refers to racemates, enantiomers and distereoisomers of these compounds and mixtures thereof.

In addition, the present disclosure embraces all geometric and positional isomers. For example, if a compound of the disclosure incorporates a double bond or a fused ring, both the cis- and trans-forms, as well as mixtures, are embraced within the scope of the disclosure.

Within the present disclosure it is to be understood that a compound of the disclosure may exhibit the phenomenon of tautomerism and that the formulae drawings within this specification can represent only one of the possible tautomeric forms. It is to be understood that the disclosure encompasses any tautomeric form and is not to be limited merely to any one tautomeric form utilized within the formulae drawings.

It is also to be understood that certain compounds may exhibit polymorphism, and that the disclosure encompasses all such forms.

In an embodiment, the compound or pharmaceutically acceptable salt thereof is a compound of formula la or a pharmaceutically acceptable salt thereof:

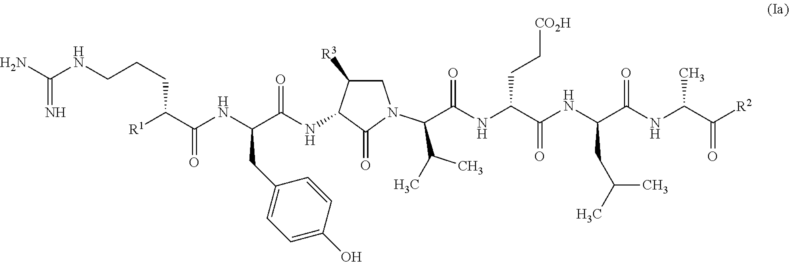

wherein R¹, R² and R³ are as defined above.

In an embodiment, the compound or pharmaceutically acceptable salt thereof is a compound of the structures depicted in Example 1 below.

In an embodiment, the compound or pharmaceutically acceptable salt thereof is a compound of one of the following structures:

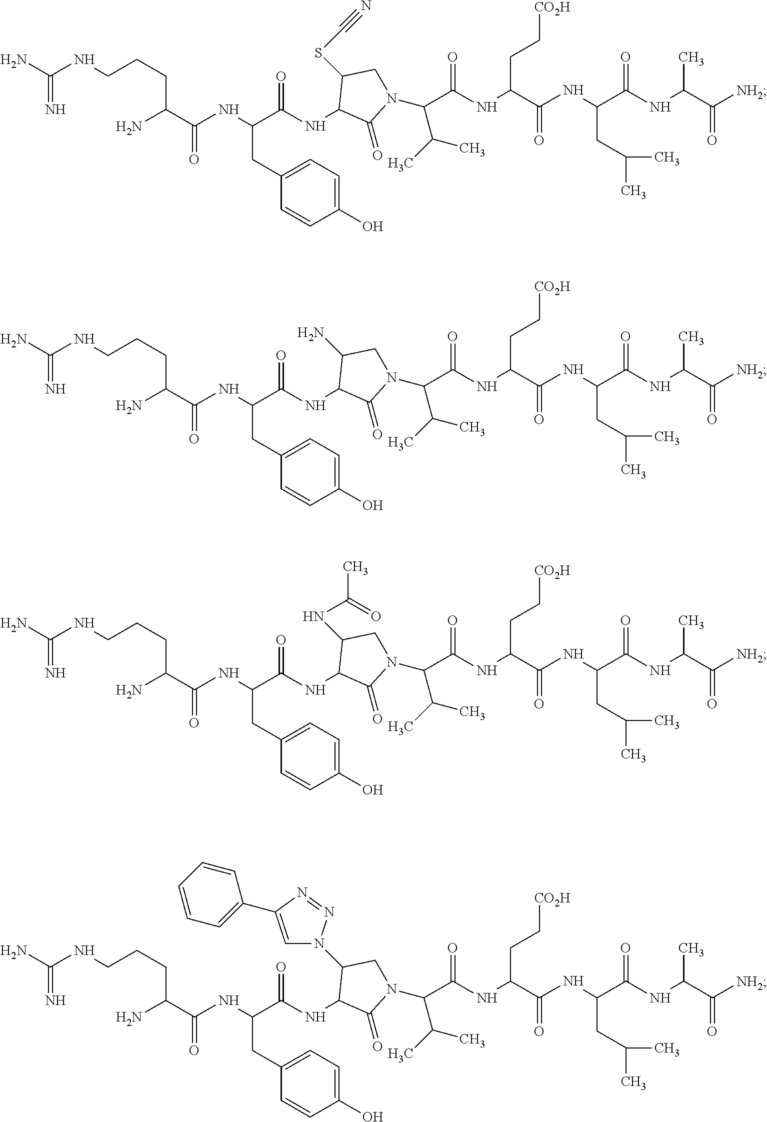 [SMILES: CC(C)CC(NC(=O)C(CCC(=O)O)NC(=O)C(C(C)C)N1CC(SC#N)C(NC(=O)C(Cc2ccc(O)cc2)NC(=O)C(N)CCCNC(=N)N)C1=O)C(=O)NC(C)C(N)=O ; CC(C)CC(NC(=O)C(CCC(=O)O)NC(=O)C(C(C)C)N1CC(N)C(NC(=O)C(Cc2ccc(O)cc2)NC(=O)C(N)CCCNC(=N)N)C1=O)C(=O)NC(C)C(N)=O ; CC(=O)NC1CN(C(C(=O)NC(CCC(=O)O)C(=O)NC(CC(C)C)C(=O)NC(C)C(N)=O)C(C)C)C(=O)C1NC(=O)C(Cc1ccc(O)cc1)NC(=O)C(N)CCCNC(=N)N ; CC(C)CC(NC(=O)C(CCC(=O)O)NC(=O)C(C(C)C)N1CC(n2cc(-c3ccccc3)nn2)C(NC(=O)C(Cc2ccc(O)cc2)NC(=O)C(N)CCCNC(=N)N)C1=O)C(=O)NC(C)C(N)=O]

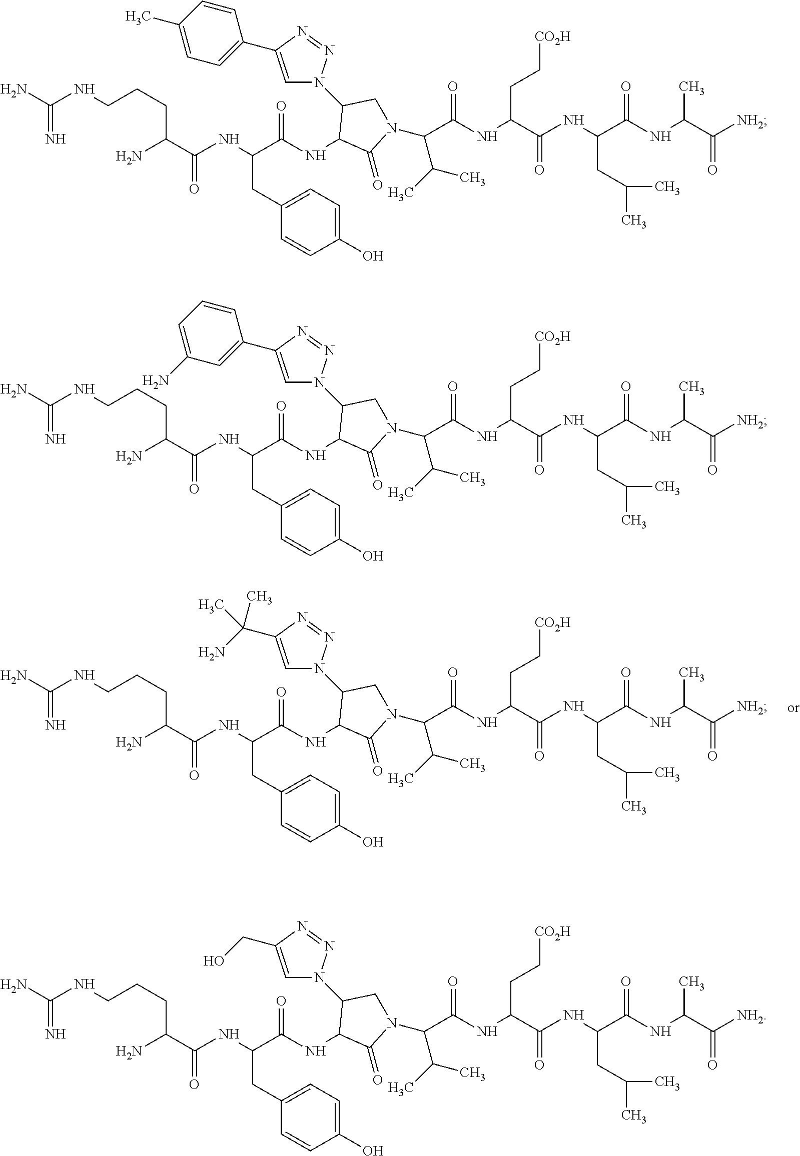

In an embodiment, the compound or pharmaceutically acceptable salt thereof is a compound of one of the following structures:

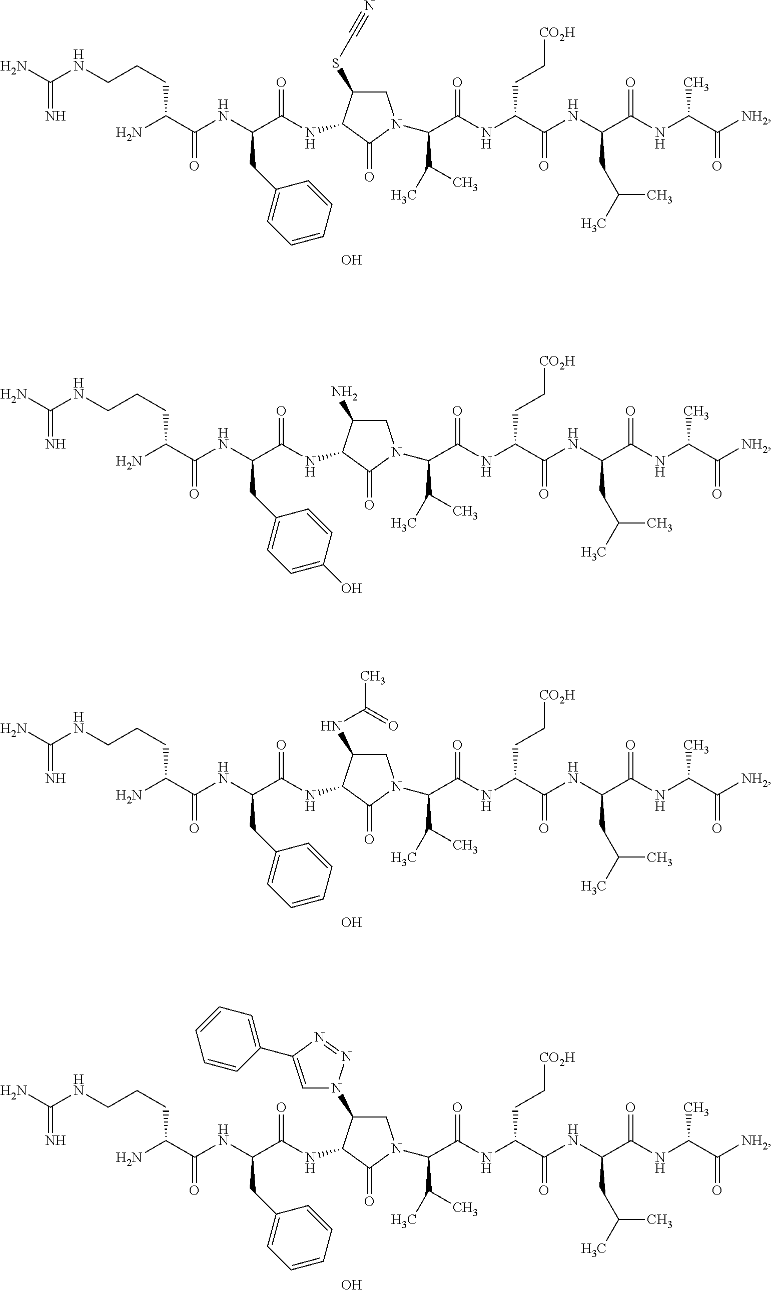

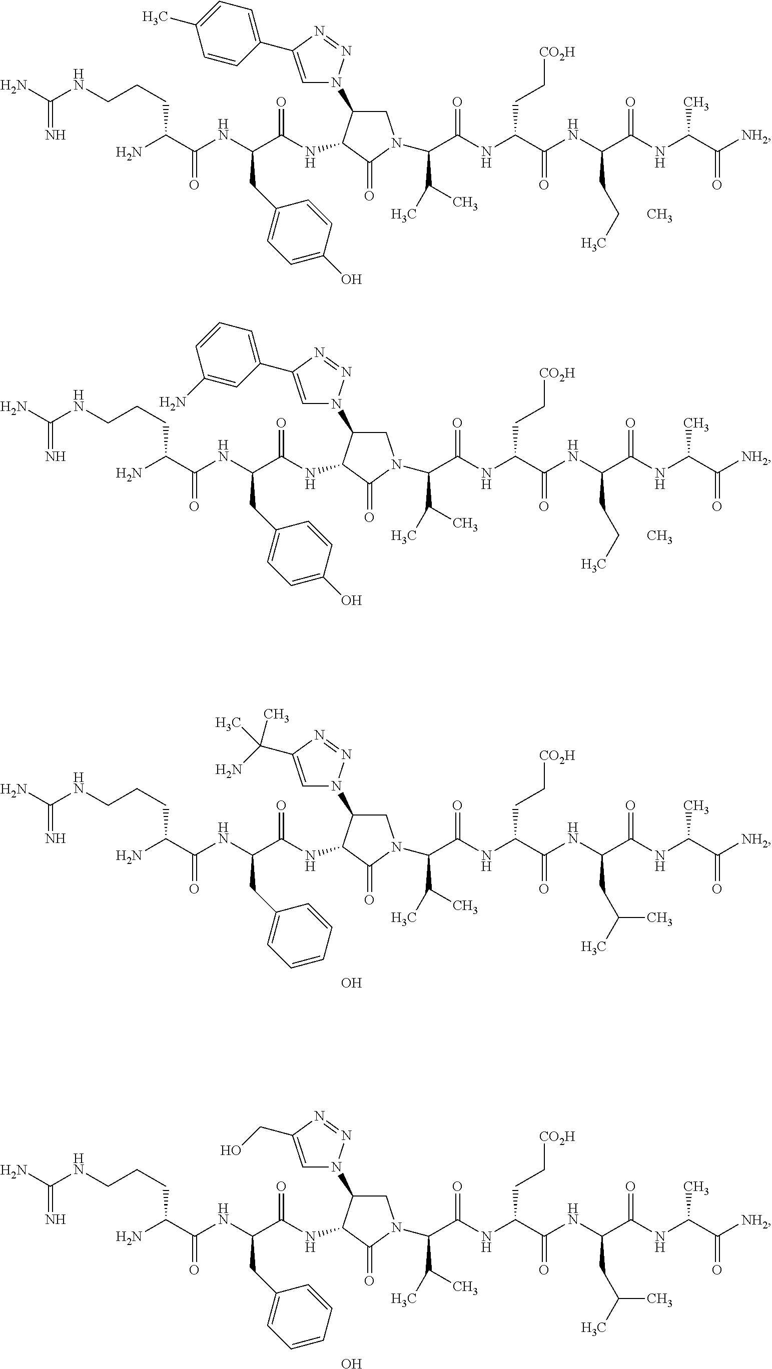

or a pharmaceutically acceptable salt thereof.

The term “pharmaceutically acceptable salt” refers to salts of the compounds described herein that are pharmacologically acceptable and substantially non-toxic to the subject to which they are administered. More specifically, these salts retain the biological effectiveness and properties of the compounds and are formed from suitable non-toxic organic or inorganic acids or bases.

For example, these salts include acid addition salts of the compounds described herein which are sufficiently basic to form such salts. Such acid addition salts include acetates, adipates, alginates, lower alkanesulfonates such as a methanesulfonates, trifluoromethanesulfonates or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like.

Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C.).

Also, where the compounds described herein are sufficiently acidic, the salts include base salts formed with an inorganic or organic base. Such salts include alkali metal salts such as sodium, lithium, and potassium salts; alkaline earth metal salts such as calcium and magnesium salts; metal salts such as aluminum salts, iron salts, zinc salts, copper salts, nickel salts and cobalt salts; inorganic amine salts such as ammonium or substituted ammonium salts, such as trimethylammonium salts; and salts with organic bases (for example, organic amines) such as chloroprocaine salts, dibenzylamine salts, dicyclohexylamine salts, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amine salts, tetramethylammonium salts, t-octylamine salts, tris-(2-hydroxyethyl)amine salts, and tris(hydroxymethyl)aminomethane salts.

Such salts can be formed quite readily by those skilled in the art using standard techniques. Indeed, the chemical modification of a pharmaceutical compound (i.e. drug) into a salt is a technique well known to pharmaceutical chemists, (See, e.g., H. Ansel et al., Pharmaceutical Dosage Forms and Drug Delivery Systems (6^{th} Ed. 1995) at pp. 196 and 1456-1457). Salts of the compounds described herein may be formed, for example, by reacting the compound with an amount of acid or base, such as an equivalent amount, in a medium such as one in which the salt precipitates or in an aqueous medium followed by lyophilization.

The present disclosure also encompasses pharmaceutically acceptable prodrug or esters of the compounds defined herein. The term “ester(s)”, as employed herein, refers to the compounds of the present disclosure or salts thereof in which hydroxy groups have been converted to the corresponding esters using, for example, inorganic or organic anhydrides, acids, or acid chlorides. The term “pharmaceutically acceptable esters” refers to esters of the compounds described herein that are pharmacologically acceptable and substantially non-toxic to the subject to which they are administered. More specifically, these esters retain the biological effectiveness and properties of the compounds described herein and act as prodrugs which, when administered in vivo, are metabolized or cleaved in such a manner as to produce the parent compounds.

Examples of esters include among others the following groups (1) carboxylic acid esters; (2) sulfonate esters, such as alkyl- or arylalkyl-sulfonate esters (for example, methanesulfonate ester); (3) phosphonate esters; (4) mono-, di- or triphosphate esters (including phosphoramidic cyclic esters); (5) carbamic acid ester (for example N-methylcarbamic ester); and (6) carbonic acid ester (for exemple methylcabonate), obtained by esterification of the hydroxy groups, the ester grouping comprising for example straight or branched chain alkyl (e.g., ethyl, n-propyl, t-butyl, n-butyl, methyl, propyl, isopropyl, butyl, isobutyl or pentyl), alkoxyalkyl (e.g., methoxymethyl, acetoxymethyl and 2,2-dimethylpropionyloxymethyl), arylalkyl (e.g., benzyl), aryloxyalkyl (e.g., phenoxymethyl), aryl (e.g., phenyl optionally substituted with, for example, halogen, C_{1.4} alkyl, or C₁-4 alkoxy or amino). Further information concerning the preparation and use of esters for the delivery of pharmaceutical compounds is available in Design of Prodrugs. Bundgaard H ed. (Elsevier, 1985). See also, H. Ansel et al., Pharmaceutical Dosage Forms and Drug Delivery Systems (6^{th} Ed. 1995) at pp. 108-109; Krogsgaard-Larsen, et. al., Textbook of Drug Design and Development (2^{nd} Ed. 1996) at pp. 152-191; Jarkko Rautio et al., Nat. Rev. Drug Discov., 7, pp. 255-270 (2008); and Pen-Wei Hsieh et al., Curr. Pharm. Des., 15(19), pp. 2236-2250 (2009).

The compounds described herein may be esterified by a variety of conventional procedures including reacting the appropriate anhydride, carboxylic acid or acid chloride with the alcohol group of a compound described herein. For example, an appropriate anhydride may be reacted with an alcohol in the presence of a base, such as 1,8-bis[dimethylamino]naphthalene or N,N-dimethylaminopyridine, to facilitate acylation. Also, an appropriate carboxylic acid can be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and, optionally, an acylation catalyst. Esterification can also be effected using the appropriate carboxylic acid. Reaction of an acid chloride with the alcohol can also be carried out. When a compound described herein contains a number of free hydroxy groups, those groups not being converted into a prodrug functionality may be protected (for example, using a t-butyl-dimethylsilyl group), and later deprotected. Also, enzymatic methods may be used to selectively phosphorylate or dephosphorylate alcohol functionalities. One skilled in the art would readily know how to successfully carry out these as well as other known methods of esterification of alcohols.

The compounds or pharmaceutically acceptable salts thereof described herein may exist in unsolvated as well as solvated forms with solvents such as water (hydrates), ethanol (ethanolates), and the like, and it is intended that the invention embrace both solvated and unsolvated forms.

The compounds or pharmaceutically acceptable salts thereof described herein may be modified to confer them with desirable properties, for example to improve their stability once administered to a subject (i.e., once administered it has a longer half-life or longer period of effectiveness as compared to the unmodified form). Such modifications are well known to those skilled in the art (e.g., polyethylene glycol derivatization (PEGylation), microencapsulation, glycosylation, etc.).

The compounds of the present disclosure may be obtained by any method of peptide synthesis known to those skilled in the art, e.g., exclusive solid phase synthesis, partial solid phase synthesis, fragment condensation, classical solution synthesis. For example, the compounds can be obtained by solid phase peptide synthesis, which in brief, consists of coupling the carboxyl group of the C-terminal amino acid to a resin (e.g., benzhydrylamine resin, chloromethylated resin, hydroxymethyl resin) and successively adding N-alpha protected amino acids. The protecting groups maybe any such groups known in the art. Before each new amino acid is added to the growing chain, the protecting group of the previous amino acid added to the chain is removed. Such solid phase synthesis has been described, for example, by Merrifield, 1964, J. Am. Chem. Soc. 85: 2149; Vale et al., 1981, Science, 213: 1394-1397, in U.S. Patent Nos. 4,305,872 and 4,316,891, Bodonsky et ah, 1966, Chem. Ind. (London), 38:1597; Pietta and Marshall, 1970, Chem. Comm. 650. The coupling of amino acids to appropriate resins is also well known in the art and has been described in U.S. Pat. No. 4,244,946. (Reviewed in Houver-Weyl, Methods of Organic Chemistry. Vol E22a. Synthesis of Peptides and peptidomimetics, Murray Goodman, Editor-in-Chief, Thieme. Stuttgart. New York 2002).

During any process of the preparation of a compound of the present disclosure, it may desirable to protect sensitive reactive groups on any of the molecule concerned. This may be achieved by means of conventional protecting groups such as those described in Protective Groups In Organic Synthesis by T.W. Greene & P. G. M. Wuts, 1991, John Wiley and Sons, New-York; and Peptides: chemistry and Biology by Sewald and Jakubke, 2002, Wiley- VCH, Wheinheim p.142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (For Thr and Ser), Cbz (Tyr, Thr, Ser, Arg, Lys), methyl ethyl, cyclohexyl (Asp, His), Boc (Arg, His, Cys) etc. The protecting groups may be removed at a convenient subsequent stage using methods known in the art.

In one embodiment, the compounds disclosed herein may generally be synthesized according to the FMOC protocol in an organic phase with protective groups. They can be purified with a yield of 70% with HPLC on a C₁₈ column and eluted with an acetonitrile gradient of 10-60%. Their molecular weight can then be verified by mass spectrometry (Reviewed in Fields, G.B. “Solid-Phase Peptide Synthesis”. Methods in Enzymology. Vol. 289, Academic Press, 1997).

Purification of the synthesized compounds is carried out by standard methods, including chromatography (e.g., ion exchange, size exclusion, affinity), centrifugation, precipitation or any standard technique for the purification of peptides and peptide derivatives. In one embodiment, thin-layered chromatography is employed. In another embodiment, reverse phase HPLC is employed. Other purification techniques well known in the art and suitable for peptide isolation and purification may be used in the present disclosure.

Where the processes for the preparation of the compounds according to the present disclosure give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques such as the formation of diastereoisomeric pairs by salt formation with an optically active acid followed by fractional crystallization and regeneration of the free base. The compounds may also be resolved by formation of diastereomeric esters or amides, followed by removal of the chiral auxiliary. Alternatively, the compounds may be resolved using chiral HPLC column.

The present disclosure also provides a pharmaceutical composition comprising the compounds or pharmaceutically acceptable salts thereof disclosed herein in combination with a pharmaceutically acceptable carrier.

The term carrier refers to diluents, adjuvants, excipients such as a filler or a binder, a disintegrating agent, a lubricant a silica flow conditioner a stabilizing agent or vehicles with which the compounds or pharmaceutically acceptable salts are administered. Such pharmaceutical carriers include sterile liquids such as water and oils including mineral oil, vegetable oil (e.g., peanut oil, soybean oil, sesame oil, canola oil), animal oil or oil of synthetic origin. Aqueous glycerol and dextrose solutions as well as saline solutions may also be employed as liquid carriers of the pharmaceutical compositions of the present disclosure. Of course, the choice of the carrier depends on the nature of the compounds or pharmaceutically acceptable salts, its solubility and other physiological properties as well as the target site of delivery and application. For example, carriers that can penetrate the blood brain barrier are used for treatment, prophylaxis or amelioration of symptoms of diseases or conditions (e.g., inflammation) in the central nervous system. Examples of suitable pharmaceutical carriers are described in Remington: The Science and Practice of Pharmacy by Alfonso R. Gennaro, 2003, 21^{st} edition, Mack Publishing Company.

Further pharmaceutically suitable materials that may be incorporated in pharmaceutical preparations of the present disclosure include absorption enhancers, pH regulators and buffers, osmolarity adjusters, preservatives, stabilizers, antioxidants, surfactants, thickeners, emollient, dispersing agents, flavoring agents, coloring agents and wetting agents.

Examples of suitable pharmaceutical excipients include water, glucose, sucrose, lactose, glycol, ethanol, glycerol monostearate, gelatin, rice, starch flour, chalk, sodium stearate, malt, sodium chloride and the like. The pharmaceutical compositions of the present disclosure can take the form of solutions, capsules, tablets, creams, gels, powders sustained release formulations and the like. The composition can be formulated as a suppository, with traditional binders and carriers such as triglycerides. Such compositions contain a therapeutically effective amount of the therapeutic composition, together with a suitable amount of carrier so as to provide the form for proper administration to the subject. The formulations are designed so as to suit the mode of administration and the target site of action (e.g., a particular organ or cell type).

Examples of fillers or binders that may be used in accordance with the present disclosure include acacia, alginic acid, calcium phosphate (dibasic), carboxymethylcellulose, carboxymethylcellulose sodium, hydroxyethylcellulose, hydroxypropylcellulose, hydroxypropylmethylcellulose, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth micro crystalline cellulose, starch, and zein. In certain embodiments, a filler or binder is microcrystalline cellulose.

Examples of disintegrating agents that may be used include alginic acid, carboxymethylcellulose, carboxymethylcellulose sodium, hydroxypropylcellulose (low substituted), microcrystalline cellulose, powdered cellulose, colloidal silicon dioxide, sodium croscarmellose, crospovidone, methylcellulose, polacrilin potassium, povidone, sodium alginate, sodium starch glycolate, starch, disodium disulfite, disodium edathamil, disodium edetate, disodiumethylenediaminetetraacetate (EDTA) crosslinked polyvinylpyrollidines, pregelatinized starch, carboxymethyl starch, sodium carboxymethyl starch, microcrystalline cellulose.

Examples of lubricants include calcium stearate, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine.

Examples of silica flow conditioners include colloidal silicon dioxide, magnesium aluminum silicate and guar gum. Another most preferred silica flow conditioner consists of silicon dioxide.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

The pharmaceutical composition of the present disclosure may be administered by any suitable route including, intravenous or intramuscular injection, intraventricular or intrathecal injection (for central nervous system administration), orally, topically, subcutaneously, subconjunctivaly, or via intranasal, intradermal, sublingual, vaginal, rectal or epidural routes.

Other delivery system well known in the art can be used for delivery of the pharmaceutical compositions of the present disclosure, for example via aqueous solutions, encapsulation in microparticles, or microcapsules.

The compounds or pharmaceutically acceptable salts thereof, or pharmaceutical composition comprising same, may be useful for inhibiting the activity of an IL-1 receptor (IL-1R) in a cell, either in vivo or in vitro.

Such inhibition of the activity of an IL-1R may be useful for the treatment of diseases or conditions in which abnormal or dysregulated IL-1R activity is involved in the pathology of the disease/condition (IL-1 related disease, disorder or condition). Examples of such diseases or conditions include several inflammatory disease, disorder or condition in which the levels of interleukin-1β (IL-1β), a key cytokine mediator of inflammation, are increased.

As used herein, the term an “inflammatory disease, disorder or condition” refers to any disease, disorder, or condition in which the immune system is abnormally activated or dysregulated, and in which abnormal or dysregulated IL-1R activity is involved in the pathology of the disease/condition, for example due to increased levels/expression of IL-1B. In some embodiments, an inflammatory disease, disorder, or condition that can be treated according to the present disclosure is inflammation of the upper and lower respiratory tract, for example, bronchial asthma, allergic asthma, non-allergic asthma, lymphomatous tracheobronchitis, allergic hypersensitivity or a hypersecretion condition, such as chronic bronchitis and cystic fibrosis; pulmonary fibrosis of various aetiologies (e.g., idiopathic pulmonary fibrosis), chronic obstructive pulmonary disease (COPD), sarcoidosis, allergic and non-allergic rhinitis; allergic or non-allergic urticaria; a skin-related diseases characterized by deregulated inflammation, tissue remodeling, angiogenesis, a disease of the gastrointestinal tract, such as Crohn's disease, ulcerative colitis, Hirschsprung's disease, diarrhea, malabsorption conditions; strokes, emesis; a disease of the immune system, such as in the splenic and lymphatic tissues, an autoimmune disease or other immune-related diseases; a disease of the cardiovascular system, such as pulmonary edema, hypertension, atherosclerosis, pre-eclampsia, complex regional pain syndrome type 2, stroke and chronic inflammatory diseases such as arthritis, a bone-related diseases such as rheumatoid arthritis, as well as pain, chronic pain such as fibromyalgia, hypoxic-ischemic newborn brain injuries, and ischemic diseases of the retina (retinopathies), such as retinopathy of prematurity (ROP), diabetic retinopathy (DR) and age-related macular degeneration (AMD).

Additional examples of inflammatory disorders include acne vulgaris; acute respiratory distress syndrome; Addison's disease; allergic intraocular inflammatory diseases, ANCA-associated small-vessel vasculitis; ankylosing spondylitis; atopic dermatitis; autoimmune hemolytic anemia; autoimmune hepatitis; Behcet's disease; Bell's palsy; bullous pemphigoid; cerebral ischaemia; cirrhosis; Cogan's syndrome; contact dermatitis; Cushing's syndrome; dermatomyositis; diabetes mellitus; discoid lupus erythematosus; lupus nephritis; eosinophilic fasciitis; erythema nodosum; exfoliative dermatitis; focal glomerulosclerosis; focal segmental glomerulosclerosis; segmental glomerulosclerosis; giant cell arteritis; gout; gouty arthritis; graft-versus-host disease (GvHD); hand eczema; Henoch-Schonlein purpura; herpes gestationis; hirsutism; idiopathic cerato-scleritis; idiopathic thrombocytopenia purpura; immune thrombocytopenia purpura inflammatory bowel or gastrointestinal disorders, inflammatory dermatoses; lichen planus; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; septic shock syndrome; shoulder tendinitis or bursitis; Sjogren's syndrome; Still's disease; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

IL-1β has been identified as a key inducer of inflammation in pre-term birth (PTB) by binding to its ubiquitously expressed receptor IL-1RI, thus promoting activation and amplification of the inflammatory cascade in utero. Inflammatory cytokines such as IL-1β in the fetal circulation rapidly spread and affect organs that are particularly vulnerable to inflammatory stressors at an early stage of development, such as lung, intestine, and brain. Thus, the compounds or pharmaceutically acceptable salts thereof, or pharmaceutical composition comprising same, may be useful for preventing PTB and/or to prevent or reduce of the risk of perinatal or neonatal morbidity and mortality caused by antenatal fetal inflammation. Perinatal/neonatal morbidity comprises organ damages, including damages to the lungs, the brain and/or the intestines, respiratory problems (asphyxia, bronchopulmonary dysplasia, pneumonia), immune system problems, gastrointestinal problems (e.g., necrotizing enterocolitis), systemic and pulmonary hypertension, early onset neonatal sepsis, septic shock, and/or neurological or developmental problems/handicaps. The neurological and/or developmental problems in the newborns may result in short-, mid- and/or long-term neurological conditions, complications or sequelae, such as cerebral palsy, impaired cognitive skills, behavioral and psychological problems (e.g., mental deficiency and autism). Thus, the term “reducing perinatal/neonatal morbidity” or “reducing the risk of perinatal/neonatal morbidity” encompasses reducing direct damages, injuries and disorders of the fetus or newborn, but also long-term complications/sequelae thereof that may occur later during childhood/adulthood (or reducing the risk of developing such disorders/complications). The prevention of PTB and/or the prevention or reduction of the risk of perinatal or neonatal morbidity and mortality caused by antenatal fetal inflammation is achieved by administering the compounds or pharmaceutically acceptable salts thereof, or pharmaceutical composition comprising same to an expectant mother.

As used herein, the term “treat”, “treatment” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The compounds or pharmaceutically acceptable salts thereof described herein may be administered alone or in combination with other active agents useful for the treatment, prophylaxis or amelioration of symptoms of an IL-1-associated disease or condition, such as an inflammatory disease or condition. Thus, the compositions and methods of the present disclosure may be used in combination with other agents exhibiting the ability to modulate IL-1 activity (e.g., synthesis, release and/or binding to IL-1R/IL-1RacP) or to reduce the symptoms of an IL-1 associated disease (e.g., rheumatoid arthritis and inflammatory bowel disease). Example of such agents include but are not limited to antirheumatic drugs such as chloroquine, auranofm (Ridaura™), dexamethasone, sodium aurothiomalate, methotrexate (see Lee et ah, 1988, Proc. Int. Acad. Sci, 85:1204), probucol (see Ku et al, 1988, Am. J. Cardiol. 62:778), pentoxyfylline (e.g., Sullivan et al, 1988, Infect. Immun. 56 : 1722), disulfiram (see Marx 1988, Science, 239:257), antioxidants such as nordihydroguaiaretic acid (lee et al, 1988, Int J. Immunopharm., 10:385), IL-I Trap (see e.g., 2003, Curr. Opin. Inv. Drugs, 4(5): 593-597), Anakinra (Kineret™, PCT Application WO00236152), leflunomide, corticosteroids (Medrol™, Deltasone™, Orasone™) as well as other agents such as those described in Bender and Lee (1989) Annual Reports in Medicinal Chemistry, chapter 20: Pharmacological Modulation of IL-1: 185-193). Other drugs may also be used in combination with the compounds disclosed herein like anti-inflammatory drugs such as Non-Steroidal Antiinflammatory Drugs (NSAIDS, e.g., Rofecoxib (VIOXX™), Celecoxib (Celebrex™), Valdecoxib (Bextra™), Aspirin™, Advil™), anti TNF-a drugs (Infliximab, etanercept, adalimumab), collagenase inhibitors, tocolytic agents such as betamimetics (ritodrine, terbutaline, hexoprenaline, salbutamol), calcium channel blockers (nifedipine, nicardipine), oxytocin receptor blockers (atosiban, barusiban), and prostaglandin inhibitors (indomethacin, sulindac, celecoxib), and others. Of course, a combination of two or more compounds or pharmaceutically acceptable salts thereof and their combination with one or more drug can also be used, in all combinations.

### MODE(S) FOR CARRYING OUT THE INVENTION

The present invention is illustrated in further details by the following non-limiting examples.

### Example 1

### Materials and Methods

### General Chemistry Methods

Unless otherwise specified, all non-aqueous reactions were performed under an inert argon atmosphere. All glassware was dried with a flame and a flushing stream of argon gas or stored in the oven and let cool under an inert atmosphere prior to use. Anhydrous solvents (THF, DCM, MeCN, MeOH and DMF) were obtained by passage through solvent filtration systems (Glass Contour, Irvine, CA). Anhydrous solvents were transferred by syringe. Reaction mixture solutions (after aqueous workup) were dried over anhydrous MgSO₄ or Na₂SO₄, filtered, and rotary-evaporated under reduced pressure. Column chromatography was performed on 230-400 mesh silica gel, and thin-layer chromatography was performed on alumina plates coated with silica gel (Merck 60 F₂₅₄ plates). Visualization of the developed chromatogram was performed by UV absorbance or staining with iodine or potassium permanganate solutions. Specific rotations, [α]^{D} values, were calculated from optical rotations measured at 25° C. in CHCl₃ at the specified concentrations (c in g/100 mL) in a 0.5 dm cell length (l) on a Anton Paar Polarimeter, MCP 200 at 589 nm, using the general formula: [α]^{D}_{25 =}(100×α)/(l×c). Nuclear magnetic resonance spectra (1H NMR, 13C NMR) were recorded on a Bruker 300 MHz spectrometer. ¹H NMR and decoupled ¹³C[¹H] NMR spectra were measured in and referenced to CDCl₃ (7.26 ppm, 77.16 ppm) as specified below. Coupling constant J values were measured in Hertz (Hz) and chemical shift values in parts per million (ppm). High resolution mass spectrometry (HRMS) data were obtained in electrospray ionization (ESI-TOF) mode by the Centre Régional de Spectrométrie de Masse de l'Université de Montréal. Protonated molecular ions [M+H]⁺, and sodium adducts [M+Na]⁺ were used for empirical formula confirmation. Analytical LCMS and HPLC analyses were performed on either a CSH-C18, 4.6×100 mm, 5 μm column with a flow rate of 0.8 mL/min or CE-C_{18 3×50} mm, 2.7 μm column with a flow rate of 0.4 mL/min using appropriate gradients from X-Y% of MeOH [0.1% formic acid (FA)] or MeCN (0.1% FA) in H₂O (0.1% FA) over 10 min: a) 10-90%, b) 50-90%, c) 30-60%, d) 5-60%, e) 30-90%, f) 20-40%.

All final peptides were purified using the respective conditions below on a Waters™ preparative HPLC instrument with UV detection at 214, 254 and 280 nm and one of the following systems: a reverse-phase Gemini™ C₁₈ column (21.2×250 mm, 5 μm) using a flow rate of 10 mL/min over 40 min; a C₁₈ Atlantis column (19×100 mm, 5 μm) using a flow rate of 24 mL/min over 15 min; a RP-Polar column (19×100 mm, 4 μm) using a flow rate of 24 mL/min over 15 min. The appropriate gradients from X-Y% of MeOH (or MeCN) containing 0.1% FA in H₂O (0.1% FA) over time were used on the following columns: A) 10-90%/30 min MeOH (0.1% FA) in H₂O (0.1% FA), Gemini™ C₁₈ column; B) 10-90%/10.0 min MeOH (0.1% FA) in H₂O (0.1% FA), Atlantis C₁₈ column; C) 30-90%/10 min MeOH (0.1% FA) in H₂O (0.1% FA), Atlantis C₁₈ column; D) 40-90%/10 min MeCN (0.1% FA) in H₂O (0.1% FA), RP-Polar column; E) 30-90%/10 min MeCN (0.1% FA) in H₂O (0.1% FA), C₁₈ Atlantis column; F) 0-50%/9 min MeOH (0.1% FA) in H₂O (0.1% FA), C₁₈ Atlantis column.

### Chemical Reagents

Unless specified otherwise, commercially available reagents were purchased from Aldrich, A & C American Chemicals Ltd., Fluka and Advanced Chemtech™ and used without further purification: copper(l)iodide, phenylacetylene, 4-ethynyltoluene, 3-ethynylaniline, 1, 1-dimethylpropargylamine (90% remainder H₂O), propargylamine, propargylalcohol, acetic acid, tris(2-carboxyethyl)phosphine hydrochloride, acetic anhydride, potassium cyanate, 1,3-bis(tert-butoxycarbonyl)-2-methyl-2-thiopseudourea, triethylamine, mercuric chloride, piperidine, DIEA, TFA, TES, TEA, HBTU, polystyrene Rink amide resin (75-100 mesh, 1%, DVB with a 0.5 mmol/g loading). All commercially available amino acids [e.g., Fmoc-D-Ala-OH, Fmoc-D-Leu-OH, Fmoc-D-Glu(t-Bu)-OH, Fmoc-D-Tyr(t-Bu)-OH, Boc-D-Arg(Pmc)-OH] were purchased from GL Biochem™ and used as received. Solvents were obtained from VWR international. Human rIL-1β (200-01B) was from PeproTech, lipopolysaccharide (LPS) Escherichia coli endotoxin (L2630) from Sigma-Aldrich, H-rytvela-NH₂ (peptide 1) from Elim Biopharmaceuticals, Hayward, CA, and Kineret® (Anakinra) from Sobi, Biovitrum Stockholm, Sweden.

Synthesis of [(3R, 4S)-4-(N₃)Agl³]-1 (2c)

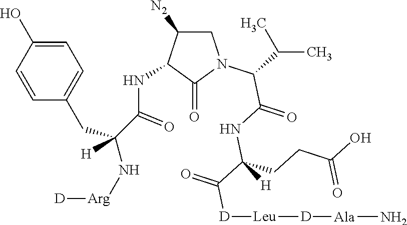

A 10-mL plastic filtration tube equipped with a polyethylene filter and stopcock was charged with polystyrene Rink amide resin (75-100 mesh, 1%, DVB with a 0.5 mmol/g loading, 100 mg, 50.0 μmol), followed by DCM (7 mL). The tube was sealed, shaken for 30 min to induce swelling and the liquid phase was removed by filtration. The Fmoc group was cleaved from the resin by treatment with a freshly prepared 20% piperidine in DMF solution (5 mL), shaking for 30 min, and removal of the liquid phase by filtration. The resin was washed repeatedly (3×per solvent) with DMF and DCM (10 mL per wash for 6 min), and the liquid phase was removed by filtration. The presence of the free amine resin was confirmed by a positive Kaiser test. Peptide elongation was conducted by treating the DMF-swollen free amine resin with a freshly prepared acylation solution composed of Fmoc-D-Ala-OH (3 eq), HBTU (3 eq), and DIEA (6 eq) in DMF (4-7 mL). After agitating for 3-5 h, at rt, the resin was filtered, the Fmoc group was cleaved as described above and peptide coupling was performed using the following sequence of acids: Fmoc-D-Leu-OH, Fmoc-D-Glu(t-Bu)-OH, N-Fmoc-(3R,4S)-4-(N₃)Agl-R-Val-OH (3c), Fmoc-D-Tyr(t-Bu)-OH, and Boc-D-Arg(Pmc)-OH. For the coupling of N-Fmoc-(3R,4S)-β-N₃-Agl-R-Val-OH (3c), only a stoichiometric quantity of dipeptide acid was used; for Fmoc-D-Tyr(t-Bu)-OH, coupling was repeated twice at higher reaction concentration. Synthetic progress was monitored using a combination of the Kaiser test and LC-MS analyses on TFA-cleaved resin aliquots, which were concentrated and dissolved in mixtures of water and MeCN. The completed peptide sequence 8c with 80% crude purity was cleaved from the resin by treatment with TFA/H2O/TES (3 mL, 95/2.5/2.5, v/v/v) with shaking for 3 h. The liquid phase was removed by filtration and collected. The resin was washed twice with TFA and the combined liquid phases were concentrated in vacuo. The residue was dissolved in a minimal volume of acetonitrile, precipitated with ice-cold diethyl ether, and centrifuged at 7000 rpm. The supernatant was removed by decantation and the precipitate was collected. The precipitation and collection processes were repeated on the supernatant. The combined white solid precipitate was dissolved in water (5 mL), freeze-dried to give a white powder (80% crude purity), and purified using method A with UV detection at 214 nm. Fractions containing pure peptide were combined and lyophilized to afford peptide [(3R,4S)-β-N₃-Agl³]-1 (2c, 9 mg, 22% yield of >95% purity): LCMS, 10-90%/10 min MeOH (0.1% FA) in water (0.1% FA), RT 8.3 min, and 10-90%/10 min MeCN (0.1% FA) in water (0.1% FA), RT 5.8 min; HRMS (ESI⁺) calcd m/z for C₃₈H₆₀N₁₄O₁₀ [M+H]⁺, 873.4690 found 873.4680.

### Synthesis of [(3S. 4S)-4-(NCS)Agl³]-1 (2d)

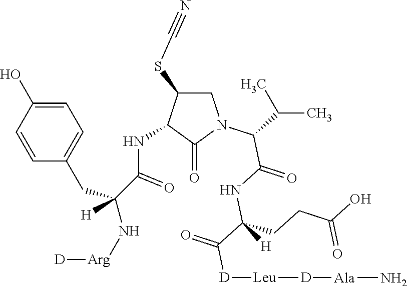

Employing N-Fmoc-(3R,4S)-4-(NCS)Agl-R-Val-OH (3d) in the representative procedure described for peptide 2c, [(3S,4S)-4-(NCS)Agl³]-1 (2d) was synthesized and purified using method A with UV detection at 214 nm (3 mg, 3% yield of >95% purity); LCMS, 10-90%/10 min MeOH (0.1% FA) in water (0.1% FA), RT 8.3 min, and 10-90%/10 min MeCN (0.1% FA) in water (0.1% FA), RT 5.8 min; HRMS (ESI⁺) calcd m/z for C₃₉H₆₀N₁₂O₁₀S [M+H]⁺, 889.4349 found 889.4342.

### Synthesis of [(3S, 4S)-4-(MeS)Agl³]-1 (2e)

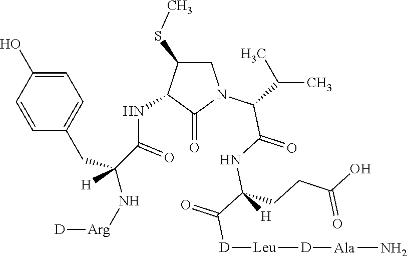

Employing N-Fmoc-(3R,4S)-4-(MeS)Agl-R-Val-OH (3e) in the representative procedure described for peptide 2c, [(3S,4S)-4-(MeS)Agl³]-1 (2e) was synthesized and purified using method A with UV detection at 214 nm (4.5 mg, 10% yield of >95% purity); LCMS 10-90%/10 min MeOH (0.1% FA) in water (0.1% FA), RT 8.7 min, and 10-90%/10 min MeCN (0.1% FA) in water (0.1% FA), RT 5.9 min; HRMS (ESI⁺) calcd m/z for C₃₉H₆₃N₁₁O₁₀S [M+H]⁺, 878.4553 found 878.4559.

### Synthesis of [(3R,4S)-4-(PhthO)Agl³]-1 (2f)

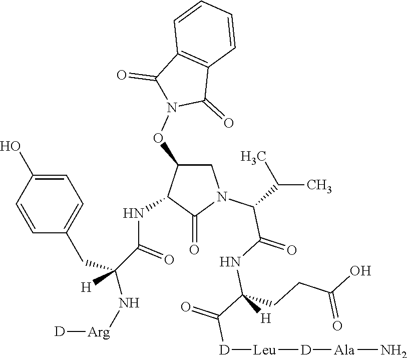

Employing N-Fmoc-(3R,4S)-4-(PhthO)Agl-R-Val-OH (3f) in the representative procedure described for peptide 2c, [(3R, 4S)-4-(PhthO)Agl³]-1 (2f) was synthesized and purified using method A with UV detection at 214 nm (5.5 mg, 11% yield of >95% purity); LCMS 30-60%/10 min MeOH (0.1% FA) in water (0.1% FA), RT 8.8 min, and 5-60%/10 min MeCN (0.1% FA) in water (0.1% FA), RT 7.3 min; HRMS (ESI⁺) calcd m/z for C₄₆H₆₄N₁₂O₁₃ [M+H]⁺, 993.4789 found 993.4786.

Synthesis of [(3R,4S)-4-(H₂NO)Agl³]-1 (2g)

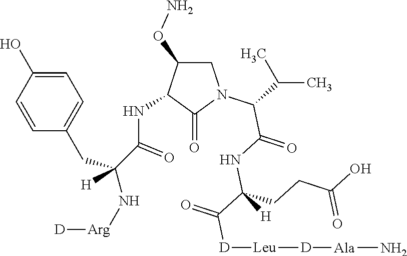

In a 10-mL plastic filtration tube equipped with a polyethylene filter, Boc-D-Arg(Pmc)-D-Tyr(t-Bu)-(3R,4S)-4-(PhthO)Agl-D-Val-D-Glu(t-Bu)-D-Leu-D-Ala-Rink amide resin 8f (100 mg, 50.0 μmol) was swollen in MeOH/DCM (1/1, v/v, 4 mL), treated with hydrazine monohydrate (73 mL, 1.50 mmol), and agitated for 5h at rt using an automated shaker (Villadsen et al. 2017). The resin was filtered, washed with DMF (3×10 mL) and DCM (3×10 mL), dried under vacuum, and stored in the fridge. Resin 8g was cleaved and the crude peptide was recovered as described for peptide 2c; LCMS analysis indicated 38% crude purity. Purification using method A with UV detection at 280 nm and collection of the pure fractions afforded [(3R,4S)-4-(H₂NO)Agl³]-1 (2g, 2.2 mg, 5% yield of >95% purity); LCMS 10-90%/10 min MeOH (0.1% FA) in water (0.1% FA), RT 6.6 min, and 10-90%/10 min MeCN (0.1% FA) in water (0.1% FA), RT 5.2 min; HRMS (ESI⁺) calcd m/z for C₃₈H₆₂N₁₂O₁₁ [M+H]⁺, 863.4661 found 863.4687.

Synthesis of [(3R,4S)-4-(H₂N)Agl³]-1 (2h)

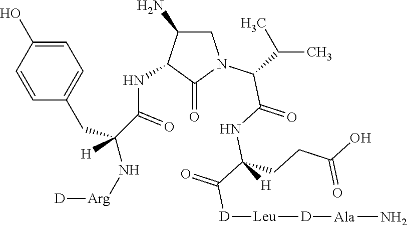

In a 10-mL plastic filtration tube equipped with a polyethylene filter, Boc-D-Arg(Pmc)-D-Tyr(t-Bu)-(3R,4S)-4-(N₃)Agl-D-Val-D-Glu(t-Bu)-D-Leu-D-Ala-Rink amide resin 8c (100 mg, 50.0 μmol) was swollen in THF/H₂O (9/1, v/v, 4 mL), treated with tris(2-carboxyethyl)phosphine hydrochloride (40 μL, 150 μmol), and agitated for 4h at rt on an automated shaker (Pandey et al. 2013). The resin was filtered, washed with DMF (3×10 mL), MeOH (3×10 mL), THF (3×10 mL), and DCM (3×10 mL), dried under vacuum, and stored in the fridge. Resin 8h was cleaved and the crude peptide was recovered as described for peptide 2c; LCMS analysis indicated 70% crude purity. Purification using method A with UV detection at 280 nm, and collection and free-drying of the pure fractions afforded [(3R,4S)-4-(H₂N)Agl³]-1 (2h, 7.6 mg, 18% yield of >95% purity): LCMS 10-90%/10 min MeOH (0.1% FA) in water (0.1% FA), RT 5.8 min, and 50-90%/10 min MeCN (0.1% FA) in water (0.1% FA), RT 1.0 min; HRMS (ESI⁺) calcd m/z for C₃₈H₆₂N₁₂₀₁₀ [M+H]⁺, 847.4785 found 847.4780.

### Synthesis of [(3R,4S)-4-(AcHN)Agl³]-1 (2i)

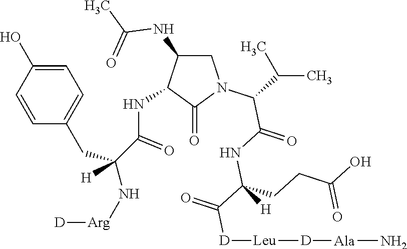

A 10-mL plastic filtration tube equipped with a polyethylene filter was charged with Boc-D-Arg(Pmc)-D-Tyr(t-Bu)-(3R,4S)-4-(H₂N)Agl-D-Val-D-Glu(t-Bu)-D-Leu-D-Ala-Rink amide resin 8h (100 mg, 50.0 μmol), which was swollen in anhydrous DMF (2.00 mL) at rt, treated with acetic anhydride (14 mL, 150 mmol) followed by DIEA (52 μL, 300 μmol), and agitated at rt for 3 h. Water (0.5 mL) was added to the tube, which was agitated for 30 min. The resin was filtered, washed with DMF (3×10 mL) and DCM (3×10 mL), dried under vacuum, and stored in the fridge. Resin 9i was cleaved and the crude peptide was recovered as described for peptide 2c; LCMS analysis indicated 41% crude purity. Purification using method A with UV detection at 214 nm, and collection and free-drying of the pure fractions afforded [(3R,4S)-4-(AcHN)Agl³]-1 (2i, 4 mg, 9% yield of >95% purity); LCMS 10-90%/10 min MeOH (0.1% FA) in water (0.1% FA), RT 7.5 min, and 10-90%/10 min MeCN (0.1% FA) in water (0.1% FA), RT 5.9 min; HRMS (ESI⁺) calcd m/z for C₄₀H₆₄N₁₂O₁₁ [M+H]⁺, 890.4850 found 890.4867.

Synthesis of [(3R,4S)-4-(H₂N(C═O)HN)Agl³]-1 (2j)

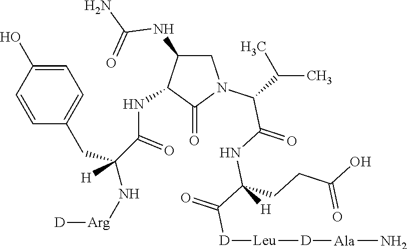

A 10-mL plastic filtration tube equipped with a polyethylene filter was charged with Boc-D-Arg(Pmc)-D-Tyr(t-Bu)-(3R,4S)-4-(H₂N)Agl-D-Val-D-Glu(t-Bu)-D-Leu-D-Ala-Rink amide resin 8h (100 mg, 50.0 μmol), which was swollen in THF (2.00 mL) at rt, treated with potassium cyanate (10 mg, 150 mmol) followed by AcOH (8 μL, 150 μmol) and H₂O (0.1 mL), and agitated at rt for 4 h on an automated shaker. The resin was filtered, washed with DMF (3×10 mL) and DCM (3×10 mL), dried under vacuum, and stored in the fridge. Resin 9j was cleaved and the crude peptide was recovered as described for peptide 2c; LCMS analysis indicated 57% purity. Purification using method A with UV detection at 214 nm, and collection and free-drying of the pure fractions afforded [(3R,4S)-4-(H₂N(C═O)HN)Agl³]-1 (2j, 4 mg, 8% yield of >95% purity); LCMS 10-90%/10 min MeOH (0.1% FA) in water (0.1% FA), RT 7.3 min, and 10-90%/10 min MeCN (0.1% FA) in water (0.1% FA), RT 5.1 min; HRMS (ESI⁺) calcd m/z for C₃₉H₆₃N₁₃O11 [M+H]⁺, 890.4843 found 890.4841.

Synthesis of [(3R,4S)-4-(H₂N(C═NH)HN)Agl³]-1 (2k)

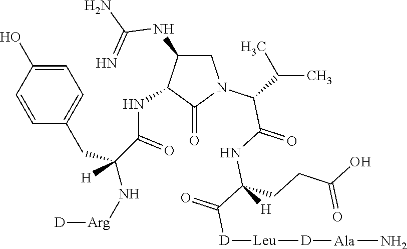

A 10-mL plastic filtration tube equipped with a polyethylene filter was charged with Boc-D-Arg(Pmc)-D-Tyr(t-Bu)-(3R,4S)-4-(H₂N)Agl-D-Val-D-Glu(t-Bu)-D-Leu-D-Ala-Rink amide resin 8h (100 mg, 50.0 μmol), and washed with DMF (×6), DCM (×6), dry DCM (×3) and dry DMF (×6). The resin was swollen in dry DMF (4 mL), treated with 1,3-bis(tert-butoxycarbonyl)-2-methyl-2-thiopseudourea (22 mL, 75 mmol) and triethylamine (63 mL, 450 mmol), stirred for 5 min, treated with HgCl₂ (40 mg, 150 μmol) in dry DMF (0.5 mL), and agitated for 2 h, at rt. The resin was washed with DMF (×6), and DCM (×6), dried under vacuum, and stored in the fridge. Resin 9k was cleaved and the crude peptide was recovered as described for peptide 2c; LCMS analysis indicated 62% purity. Purification using method A with UV detection at 280 nm, and collection and free-drying of the pure fractions afforded [(3R,4S)-4-(H₂N(C═NH)HN)Agl³]-1 (2k, 4 mg, 9% yield of >95% purity); LCMS 10-90%/10 min MeOH (0.1% FA) in water (0.1% FA), RT 5.9 min, and 5-60%/10 min MeCN (0.1% FA) in water (0.1% FA), RT 4.6 min; HRMS (ESI⁺) calcd m/z for C₃₉H₆₄N₁₄O10 [M+H]⁺, 889.5003 found 889.5006.

### Synthesis of [(3R,4S)-4-(4′-Phenyltriazolyl)Agl³]-1 (2l)

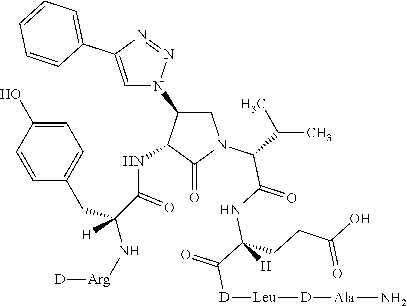

In a 10-mL plastic filtration tube equipped with a polyethylene filter, Fmoc-D-Arg(Pmc)-D-Tyr(t-Bu)-(3R,4S)-4-(N₃)Agl-D-Val-D-Glu(t-Bu)-D-Leu-D-Ala-Rink amide resin 8c (100 mg, 50.0 μmol) was swollen in anhydrous DCM (2 mL), treated with copper(I)iodide (14 mg, 75.0 μmol) and DIEA (26 μL, 150 μmol), followed by phenylacetylene (20 μL, 180 μmol) and acetic acid (9 μL, 150 μmol), and shaken at rt for 18h (Shao et al. 2011), filtered and washed with DMF (×3) and DCM (×3). Resin 10I was cleaved and the crude peptide was recovered as described for peptide 2c; LCMS analysis indicated 74% purity. Purification using method A with UV detection at 254 nm, and collection and free-drying of the pure fractions afforded [(3R,4S)-4-(4′-phenyltriazolyl)Agl³]-1 (2l, 8 mg, 17% yield of >95% purity): LCMS 10-90%/10 min MeOH (0.1% FA) in water (0.1% FA), RT 9.6 min, and 10-90%/10 min MeCN (0.1% FA) water (0.1% FA), RT 6.5 min; HRMS (ESI⁺) calcd m/z for C₄₆H₆₆N₁₄O₁₀ [M+H]⁺, 975.5159 found 975.5147.

### Synthesis of [(3R,4S)-4-(4′-p-Methylphenyltriazolyl)Agl³]-1 (2m)

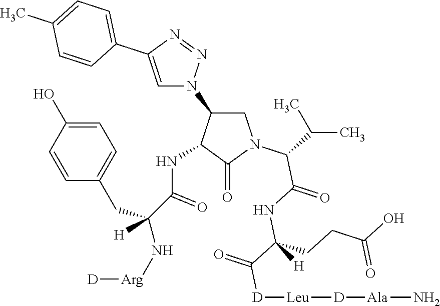

Employing the representative procedures described for the synthesis of peptide 2l, using resin 8c (100 mg, 50.0 μmol) and 4-ethynyltoluene (22.8 mL, 180 mmol), peptide 2m, was synthesized and indicated to be of 71% purity by LCMS analysis. Purification using method C with UV detection at 254 nm, and collection and free-drying of the pure fractions afforded [(3R,4S)-4-(4′-p-methylphenyltriazolyl)Agl³]-1 (2m, 7 mg, 16% yield of >95% purity); LCMS 30-90%/10 min MeOH (0.1% FA) in water (0.1% FA), RT 9.5 min, and 20-40%/10 min MeCN (0.1% FA) in water (0.1% FA), RT 6.5 min; HRMS (ESI⁺) calcd m/z for C₄₇H₆₈N₁₄O₁₀ [M+H]⁺, 989.5316 found 989.5307.

### Synthesis of [(3R,4S)-4-(4′-m-Aminophenyltriazolyl)Agl³]-1 (2n)

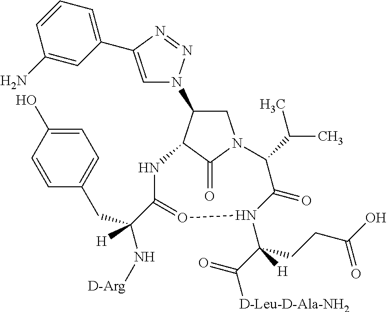

Employing the representative procedures described for the synthesis of peptide 2l, using resin 8c (100 mg, 50.0 μmol) and 3-ethynylaniline (20.3 mL, 180 mmol), peptide 2n, was synthesized and indicated to be of 69% purity by LCMS analysis. Purification using method D with UV detection at 254 nm, and collection and free-drying of the pure fractions afforded [(3R,4S)-4-(4′-m-aminophenyltriazolyl)Agl³]-1 (2n, 6 mg, 12% yield of >95% purity); LCMS 5-60%/10 min MeOH (0.1% FA) in water (0.1% FA), RT 7.9 min, and 5-60%/10 min MeCN (0.1% FA) in water (0.1% FA), RT 6.1 min; HRMS (ESI⁺) calcd m/z for C₄₆H₆₇N₁₅O₁₀ [M+H]⁺, 990.5268 found 990.5259.

### Synthesis of [(3R,4S)-4-(4′-(1,1-Dimethyl)aminomethyltriazolyl)Agl³]-1 (20)

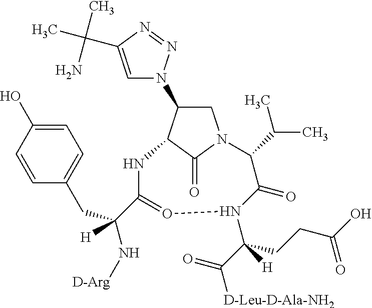

Employing the representative procedures described for the synthesis of peptide 2l, using resin 8c (100 mg, 50.0 μmol) and 1,1-dimethylpropargylamine (90% remainder H₂O, 15.0 μL, 180 umol), peptide 20 was synthesized and indicated to be of 73% purity by LCMS analysis. Purification using method B with UV detection at 214 nm, and collection and free-drying of the pure fractions afforded [(3R,4S)-4-(4′-(1, 1-dimethyl)amino-methyltriazolyl)Agl³]-1 (20, 6 mg, 13% yield of >95% purity); LCMS 10-90%/10 min MeOH (0.1% FA) in water (0.1% FA), RT 6.3 min, and 5-60%/10 min MeCN (0.1% FA) in water (0.1% FA), RT 4.9 min; HRMS (ESI⁺) calcd m/z for C₄₃H₆₉N₁₅O₁₀ [M+H]⁺, 956.5425 found 956.5408.

### Synthesis of [(3R,4S)-4-(4′-Aminomethyltriazolyl)Agl³]-1 (2p)

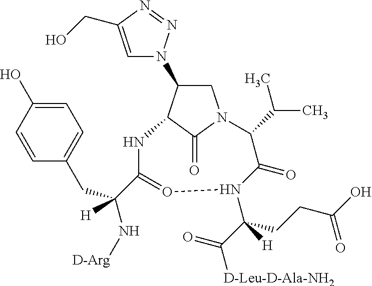

Employing the representative procedures described for the synthesis of peptide 2l, using resin 8c (100 mg, 50.0 μmol) and propargylamine (12 μL, 180 μmol), peptide 2p was synthesized and indicated to be of 53% purity by LCMS analysis. Purification using method E with UV detection at 254 nm, and collection and free-drying of the pure fractions afforded [(3R,4S)-4-(4′-aminomethyltriazolyl)Agl³]-1 (2p, 4 mg, 8% yield of >95% purity); LCMS 5-60%/10 min MeOH (0.1% FA) in water (0.1% FA), RT 7.9 min, and 5-60%/10 min MeCN (0.1% FA) in water (0.1% FA), RT 6.1 min; HRMS (ESI⁺) calcd m/z for C₄₁H₆₅N₁₅O₁₀ [M+H]⁺, 928.5039 found 928.5029.

### Synthesis of [(3R,4S)-4-(4′-Hydroxymethyltriazolyl)Agl³]-1 (2q)

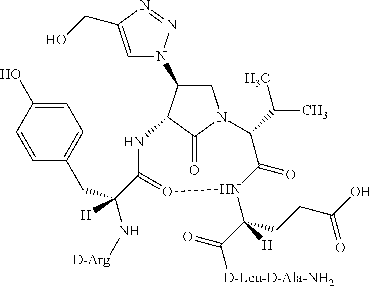

Employing the representative procedures described for the synthesis of peptide 2l, using resin 8c (100 mg, 50.0 μmol) and propargyl alcohol (7 μL, 180 μmol), peptide 2q was synthesized and indicated to be of 64% purity by LCMS analysis. Purification using method F with UV detection at 280 nm, and collection and free-drying of the pure fractions afforded [(3R,4S)-4-(4′-hydroxymethyltriazolyl)Agl³]-1 (2q, 5 mg, 10% yield of >95% purity); LCMS 5-60%/10 min MeOH (0.1% FA) in water (0.1% FA), RT 7.2 min, and 5-60%/10 min MeCN (0.1% FA) in water (0.1% FA), RT 5.73 min; HRMS (ESI⁺) calcd m/z for C₄₁H₆₄N₁₄O₁₁ [M+H]⁺, 929.4930 found 929.4952.

Synthesis of Fmoc-(3R,4S)-β-azido-Agl-(R)-Val-OH (3c)

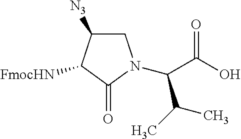

Employing the procedure described below for the synthesis of Fmoc-(3R,4S)-β-phthalimidooxy-Agl-(R)-Val-OH (3f), Fmoc-(3R,4S)-β-azido-Agl-(R)-Val-Ot-Bu (5c) (1 eq., 210 mg, 404 mmol, prepared according to Geranurimi and Lubell, 2018) was converted to Fmoc-(3R,4S)-β-azido-Agl-(R)-Val-OH (3c, 155 mg, 83%): Rf=0.07 (10% MeOH in DCM).

Synthesis of Fmoc-(3S,4S)-β-thiocyano-Agl-(R)-Val-OH (3d)

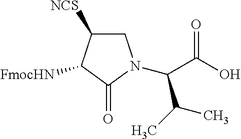

Employing the representative procedure described below for the synthesis of Fmoc-(3R,4S)-β-phthalimidooxy-Agl-(R)-Val-OH (3f), Fmoc-(3S,4S)-β-thiocyano-Agl-(R)-Val-Ot-Bu (5d, 1 eq., 55 mg, 103 μmol, prepared according to Geranurimi and Lubell, 2018) was converted to Fmoc-(3S,4S)-β-thiocyano-Agl-(R)-Val-OH (3d, 45 mg, 91%): Rf=0.1 (10% MeOH in DCM).

### Synthesis of Fmoc-(3S,4S)-β-SMe-Agl-(R)-Val-OH (3e)

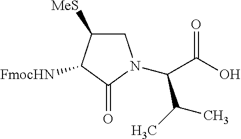

Employing the representative procedure described below for the synthesis of Fmoc-(3R,4S)-β-phthalimidooxy-Agl-(R)-Val-OH (3f), Fmoc-(3S,4S)-β-Sme-Agl-(R)-Val-Ot-Bu (5e, 1 eq., 27 mg, 51.4 mmol) was converted to Fmoc-(3S,4S)-β-Sme-Agl-(R)-Val-OH (3e, 19.0 mg, 79%): Rf=0.09 (10% MeOH in DCM).

Synthesis of Fmoc-(3R,4S)-3-phthalimidooxy-Agl-(R)-Val-OH (3f)

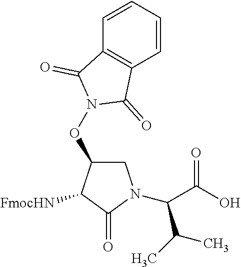

A solution of Fmoc-(3R,4S)-β-phthalimidooxy-Agl-(R)-Val-Ot-Bu (5f, 1eq., 32 mg, 50.0 mmol) in TFA (1 mL) and DCM (1 mL) was stirred at rt until TLC analysis revealed complete consumption of the ester. The volatiles were evaporated on a rotary evaporator. The residue was precipitated from ice-cooled diethyl ether and collected using a centrifuge to yield Fmoc-(3R,4S)-β-phthalimidooxy-Agl-(R)-Val-OH (3f, 26 mg, 89%), which was used without further purification: Rf 0.06 (10% MeOH in DCM).

Synthesis of tert-Butyl (3R, 4S, 2′R)-2-[3-(Fmoc)amino-4-(1,3-dioxoisoindolin-2-yl)oxy)-2-oxopyrrolidin 1-yl]-3-methylbutanoate (5f)

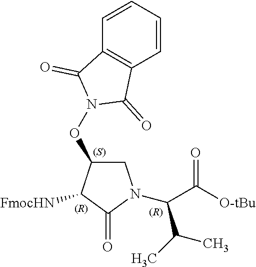

A solution of sulfamidate 4 (1 eq., 80 mg, 144 μmol, prepared according to Geranurimi and Lubell, 2018) in a mixture of DCM (2 mL) and DMF (1 mL) was treated with sodium N-hydroxyphthalimide (1 eq., 80 mg, 431 mmol), stirred at rt for 8 h, poured into 1 M NaH₂PO4, and extracted with DCM. The combined organic phase was washed with brine, dried, filtered, and evaporated to a residue, that was purified by column chromatography using a step gradient of 20-30% EtOAc in hexane. Evaporation of the collected fractions provided (4S)-phthalimide 5f (81 g, 78%) as white foam: Rr=0.37 (40% EtOAc in hexane); [a],25 16.4° (c 1, CHCI3); 1H NMR (300 MHZ, CDCl₃) δ 7.88-7.65 (m, 8H), 7.41 (t, J=7.1, 2H), 7.33 (t, J=6.9, 2H), 6.53 (d, J=8.6, 1H), 5.00 (t, J=4.5, 1H), 4.77 (dd, J=8.6, 5.1, 1H), 4.51-4.43 (m, 1H), 4.43-4.30 (m, 2H), 4.27-4.15 (m, 2H), 3.73 (dd, J=12.5, 4.3, 1H), 2.29-2.11 (m, 1H), 1.41 (s, 9H), 1.02 (d, J=6.6, 3H), 0.92 (d, J=6.8, 3H); 13C NMR (75 MHZ, CDCl₃) 0 169.4, 168.9, 163.8, 156.7, 144.0, 143.8, 141.2, 134.7, 128.8, 127.7, 127.6, 127.1, 127.0, 125.6, 125.5, 123.8, 119.8, 82.1, 67.7, 61.0, 55.0, 47.4, 47.0, 28.7, 28.0, 21.9, 19.3; HRMS (ESI-TOF) m/z [M +H]⁺ calcd for C₃₆H₃₈N₃O₈⁺640.2653, found 640.2634.

gPCR Experiments

RAW Blue cells were purchased from InvivoGen® (San Diego, CA), used at passages under 15, and cultured in DMEM growth medium supplemented with 10% serum, 50 U/mL penicillin and 200 mg/ml zeocin. Cells were grown in regular conditions (37° C., 5% CO₂), serum starved overnight, and treated with 100 ng/ML IL-1β for 4 h. Cells were respectively pre-incubated for 30 min with peptides 1 and 2 (10⁻⁶ M) or Kineret® (1.0 mg/mL) to reach equilibrium prior to the experiments (n=4 each treatment). Cells were harvested and incubated for 5 min in RIBOzol® (AMRESCO). RNA was extracted according to manufacturer's protocol and RNA concentration and integrity were measured with a NanoDrop® 1,000 spectrophotometer. A total of 500 ng of RNA was used to synthesize cDNA using iScript Reverse Transcription SuperMix® (Bio-Rad, Hercules, CA). Primers (Table 1) were designed using National Center for Biotechnology Information Primer Blast. Quantitative gene expression analysis was performed on Stratagene® MXPro3000 (Stratagene) with SYBR Green Master Mix (Bio-Rad). Gene expression levels were normalized to 18S universal primer (Ambion Life Technology, Burlington ON, Canada). Genes analyzed include IL18 and PTGHS2 [Prostaglandin H synthetase 2 or cyclooxygenase-2 (COX-2)]. Data are representative of 3 experiments (each with n=4 per treatment group).

TABLE 1
List of primers for the human genes
assessed by qPCR.

| | Forward primer | Reverse primer |
|---|---|---|
| Gene | (5′ → 3′) | (5′ → 3′) |
| IL1β | AGCTGGAGAGTGTAGATCCC | ACGGGCATGTTTTCTGCTTG |
| | AA (SEQ ID NO: 1) | (SEQ ID NO: 2) |
| PTGHS2 | ATATTGGTGACCCGTGGAGC | GTTCTCCGTACCTTCACCCC |
| | (SEQ ID NO: 3) | (SEQ ID NO: 4) |

### NF-κB QUANTI-Blue Assay

HEK-Blue IL-33/IL-1β cells (InvivoGen) were respectively pretreated with peptides 1 and 2 (10⁻⁶ M), or Kineret (1.0 mg/mL) for 30 min, followed by treatment with a constant concentration of IL-13 (100 ng/ml), and incubation at 37° C. for 4 h. Levels of secreted alkaline phosphatase in cell culture supernatant were determined using the QUANTI-Blue assay, according to the manufacturer's instructions (InvivoGen). Alkaline phosphatase activity was assessed by measuring the optical density (OD) at 620-655 nm with an EnVision Multilabel micro plate reader (PerkinElmer, Waltham, MA). Data are representative of 3 experiments (each with n=4 per treatment group).

### LPS-Induced Preterm Model in Mice

Timed-pregnant CD-1 mice at 16.5 days of gestation (G16.5) were anesthetized with 2% isoflurane and received an intraperitoneal injection of lipopolysaccharide (LPS, n=4 per group, a single dose of 10 ug) (Nadeau-Vallée et al., 2015; Kakinuma et al., 1997). A dosage of 2 mg/kg/day of peptides 1 and 2 or vehicle was respectively injected subcutaneously in the neck, every 12h until delivery. On G16.5, a dose of 1 mg/kg was injected 30 min before stimulation with LPS (to allow distribution of drugs to target tissues) and 1 mg/kg was injected 12h after stimulation (n=4 each treatment). Mice delivery was assessed every hour until term (G19-G19.5). A mouse was considered as delivering prematurely if the first pup was delivered earlier than G18.5. Data was analyzed using Prism 7 (GraphPad Software, San Diego, CA, USA) with one-way ANOVA and Dunnett's test for multiple comparisons. Outliers were detected using Grubb's test. Results were treated as significant when p was <0.05 and expressed as mean+SEM.

### Experiments in the Oxygen-Induced Retinopathy Rodent Model

Experiments in the oxygen-induced retinopathy rodent model were preformed according to the method described previously (Geranurimi et al. 2019), and summarized below.

Animals. Two-day-old (P2) Sprague Dawley rat pups and their mothers were ordered from Charles River (Raleigh, SC, USA) and acclimatized for 3 days in standard conditions. All procedures and protocols involving the use of the rats were approved by the Animal Care Committee of the research center of Hopital Maisonneuve-Rosemont and are in accordance with the Statement for the Use of Animals in Ophthalmic and Vision Research approved by the Association for Research in Vision and Ophthalmology, and guidelines established by the Canadian Council on Animal Care.

Oxygen-Induced Retinopathy in Sprague Dawley Rats. The 80% oxygen model of retinopathy was conducted as previously described (Geranurimi et al. 2019). Briefly, litters of P5 pups and their mothers were kept in a controlled 80% oxygen environment until P10. The pups were respectively injected intraperitoneally twice daily with PBS vehicle (20 μL per injection), peptide 1 or derivatives 2 (titrated to a daily dose of 2 mg/kg/day). Control litters were kept under normal air atmosphere and standard conditions. On P10, pups were euthanized by decapitation under 2% isoflurane anesthesia. Eyes were enucleated and fixed in 4% paraformaldehyde, then stored at 4° C. in phosphate-buffered saline (PBS) until further processing.

Retinal Flatmount and Immunohistochemistry. The fixed eyes were dissected, and the obtained retinas were incubated with antibodies and mounted onto slides as previously described in (Geranurimi et al. 2019). Briefly, the cornea and lens were removed from the eyes, and the retina gently removed from the underlying sclera-choroid-retinal pigmented epithelium (RPE) complex. Retinas were treated for 1h with blocking solution [1% bovine serum albumin (BSA), 1% normal goat serum, 0.1% Triton X-100 and 0.05% Tween-20 in PBS], and then incubated overnight with lectin and Iba-1 primary antibody, followed by Alexa-594-conjugated secondary antibody for 2 h. Retinas were then mounted onto microscope slides under coverslips with anti-fade mounting medium.

Microscopy. Retinal flatmounts were imaged using the Zeiss Axiolmager Z2 and the MosaiX feature of the AxioVision software as previously described (Geranurimi et al. 2019). Representative images after Iba-1 staining were taken using a laser scanning confocal microscope (Olympus IX81 with Fluoview FV1000 Scanhead) using the Fluoview Software at 30× magnification.

Quantification and Data Analysis. The FIJI software was used to quantify the area of vaso-obliteration in each retina, expressed as a percentage of the area of the whole retina. The number of Iba-1-positive cells was counted using the cell counter plug-in in the FIJI software, and the average of cell counts in 4 fields per retina was calculated. Data was analyzed using Prism 7 with one-way ANOVA and the Dunnett's test for multiple comparisons. Results were treated as significant when p was less than 0.05 and expressed as mean+SEM.

### Example 2

### Chemical Synthesis

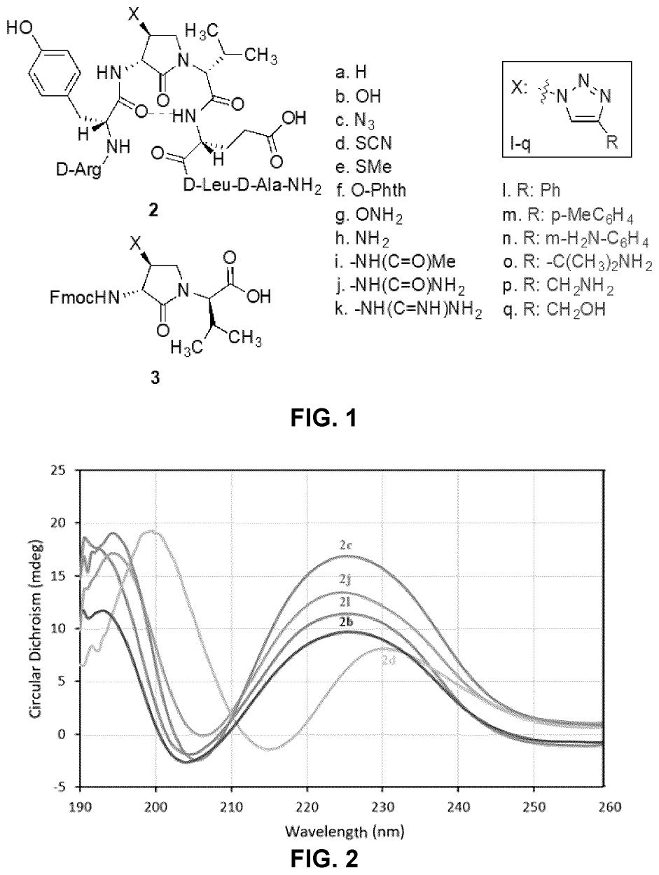

Cyclic sulfamidates are valuable intermediates for the synthesis of β-substituted amines (Meléndez and Lubell 2003). Previously, sulfamidate 4 has been employed as a bis-electrophile to prepare β-substituted-Agl residues (Geranurimi and Lubell 2018). Nucleophilic ring opening reactions of sulfamidate 4 with sodium azide and potassium thiocyanate have served in routes to prepare Fmoc-(3R,4S)-β-substituted-Agl-(R)-Val-OH analogs 3c-e with β-azido, thiocyano and methylthio ether substituents, respectively (FIG. 1) (Geranurimi and Lubell 2018; Gulea et al. 2003). The sodium salt of hydroxyphthalimide has now been used as nucleophile to open sulfamidate 4 in a 1:2 DMF/DCM mixture and provide Fmoc-(3R,4S)-β-phthalimidooxy-Agl-(R)-Val-Ot-Bu (5f) in 78% yield (Scheme 1). tert-Butyl ester 5f was converted to acid 3f using a 1:1 trifluoroacetic acid/DCM solution.

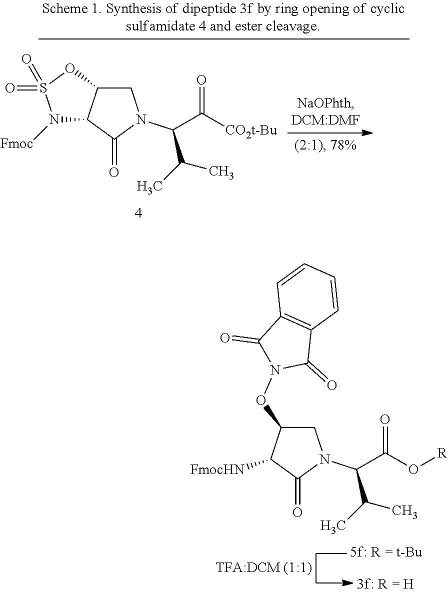

Dipeptide acids 3c-f were respectively coupled to H-D-Glu(t-Bu)-D-Leu-D-Ala-NH-Rink amide resin 6 using O-(benzotriazol-1-yl)-N,N,N′,N′-tetramethyluronium hexafluorophosphate (HBTU), and N,N-diisopropylethylamine (DIEA) in DMF to provide pentapeptide resins 7c-f. Peptide elongation by removals of Fmoc protection with 20% piperidine in DMF, and sequential couplings of Fmoc-D-Try(t-Bu)-OH and N-Boc-D-Arg(Pmc)-OH using HBTU and DIEA in DMF gave respectively protected heptapeptide resins 8c-f. Treatment of O-alkyl hydroxy phthalimide resin 8f with hydrazine monohydrate in a 1:1 MeOH/DCM mixture provided O-alkyl hydroxamine 8g (Scheme 2) (Villadsen et al. 2017). Note, O-alkyl hydroxamine 8g offers potential for the synthesis of oxime ligation conjugates (Guthrie and Proulx 2018). Resin cleavage was performed using a cocktail of 95:2.5:2.5 TFA/H₂O/TES to furnish peptides 2c-g in 38-80% crude purities. Purification by HPLC provided peptides 2c-g in 3-22% overall yields (Table 2).

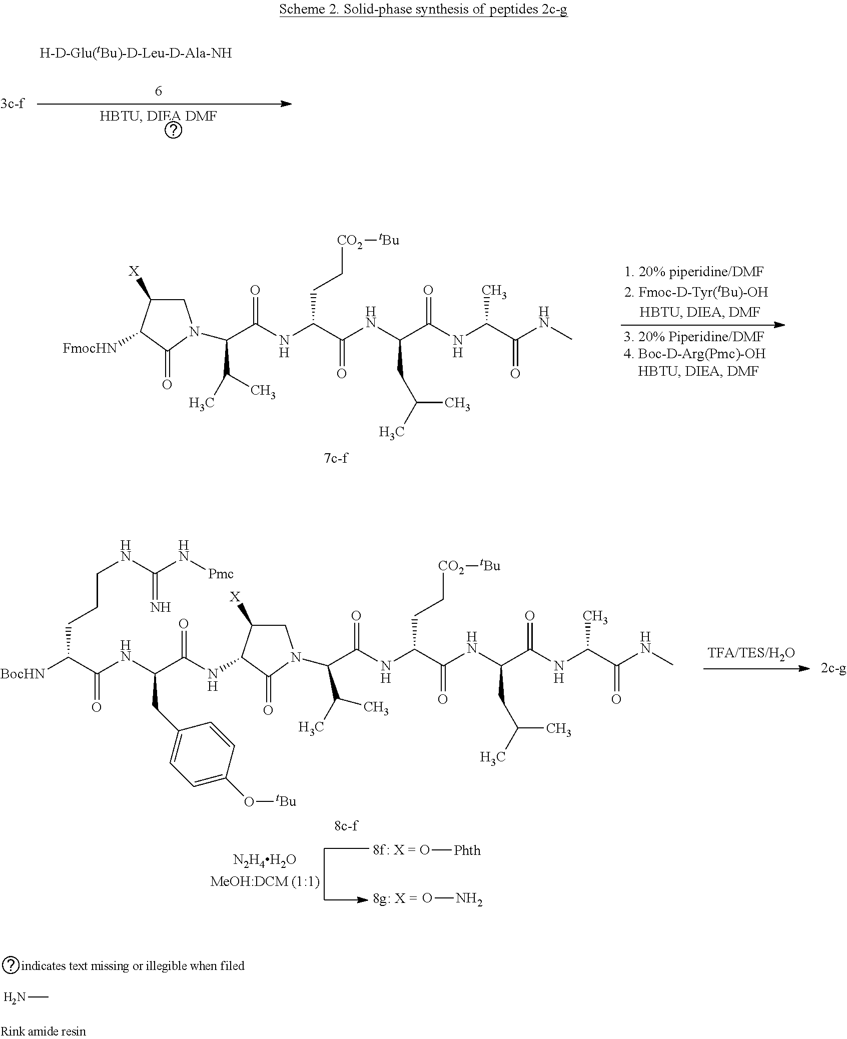

Amine resin 8h was synthesized by reduction of azide 8c using tris-(2-carboxyethyl)phosphine hydrochloride (TCEP) in a 9:1 THF: H₂O mixture (Scheme 3). Amine 8h was then employed in the synthesis of acetamide, urea and guanidine peptides 8i-k. Acetamide 8i was prepared by acylation of amine 8h using acetic anhydride and DIEA in DMF. Urea 8j was obtained from treating amine 8h with a solution of potassium cyanate and acetic acid in a 20:1 THF: H₂O mixture (Wertheim 1931). Guanidine 9k was prepared by reacting amine resin 8h with 1,3-bis(tert-butoxycarbonyl)-2-methyl-2-thiopseudourea, triethylamine and mercuric chloride in DMF (Dianati, et al. 2017). Resin cleavage and removal of the Boc and tert-butyl protection groups were concomitantly accomplished using a cocktail of 95:2.5:2.5 TFA/H₂O/TES to furnish peptides 2h-k in 41-70% crude purities. Purification by HPLC provided peptides 2h-k in 8-18% overall yields (Table 2).

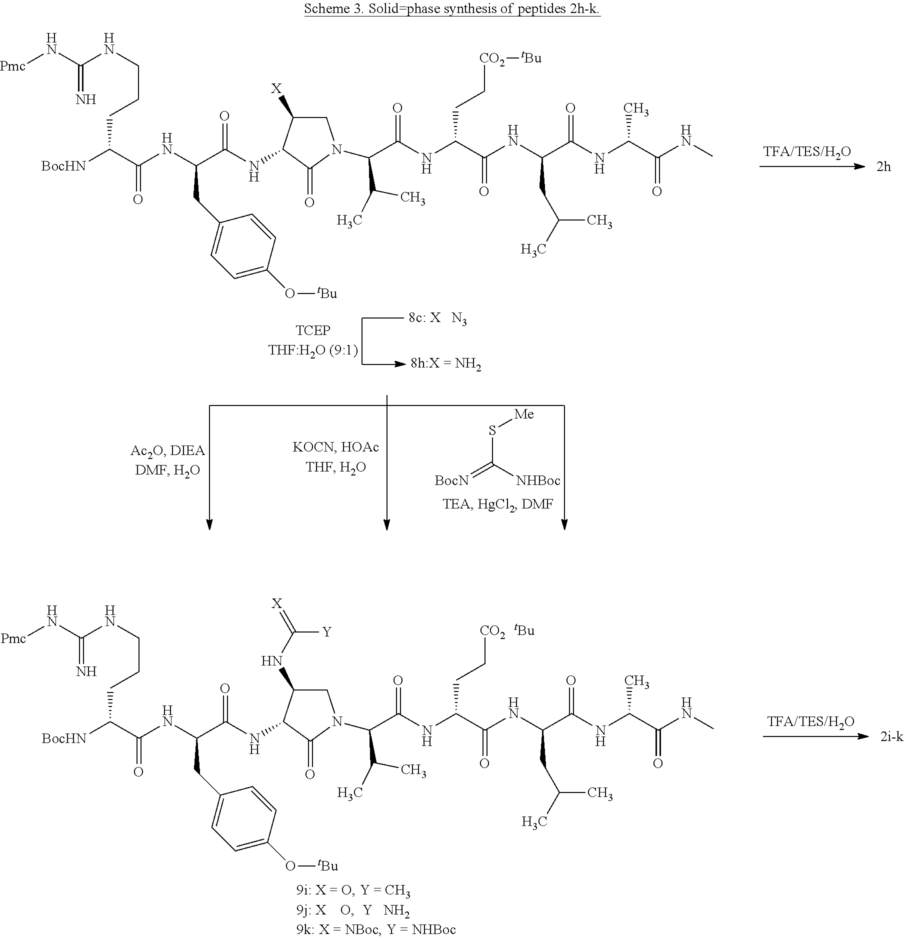

Azide 8c was also employed in CuAAC chemistry to provide 4-substituted-1,2,3-triazoles 8l-q using a set of alkynes: phenylacetylene, 4-ethynyltoluene, 3-ethynylaniline, 1,1-dimethylpropargylamine, propargylamine and propargylalcohol (Scheme 4). Alkynes were selected to study potential for aromatic, salt bridge and hydrogen bond interactions with the receptor. In the CuAAC reaction, azide 8c was treated with the corresponding alkyne, copper(I) iodide and DIEA in DCM to provide a single regioisomer, 4-substituted-1,2,3-triazoles 8l-q (Shao et al. 2011). The regioselectivity of the cyclization reaction to form 8l-q was inferred from spectral analysis of Fmoc-(3R,4R)-4-(4′-phenyltriazolyl)Agl-(R)-Val-Ot-Bu, which was synthesized from azide (3R, 4R, 2′R)-5c using identical CuAAC conditions in solution, and showed a 13C signal at 120 ppm and no signal at 133 ppm indicative of a 4-substituted triazole. As described above, resin cleavage and removal of protection were concomitantly accomplished using a TFA/H₂O/TES cocktail to furnish peptides 2l-q in 53-74% crude purities. After purification by HPLC, peptides 2l-q were isolated in 8-17% overall yields (Table 2).

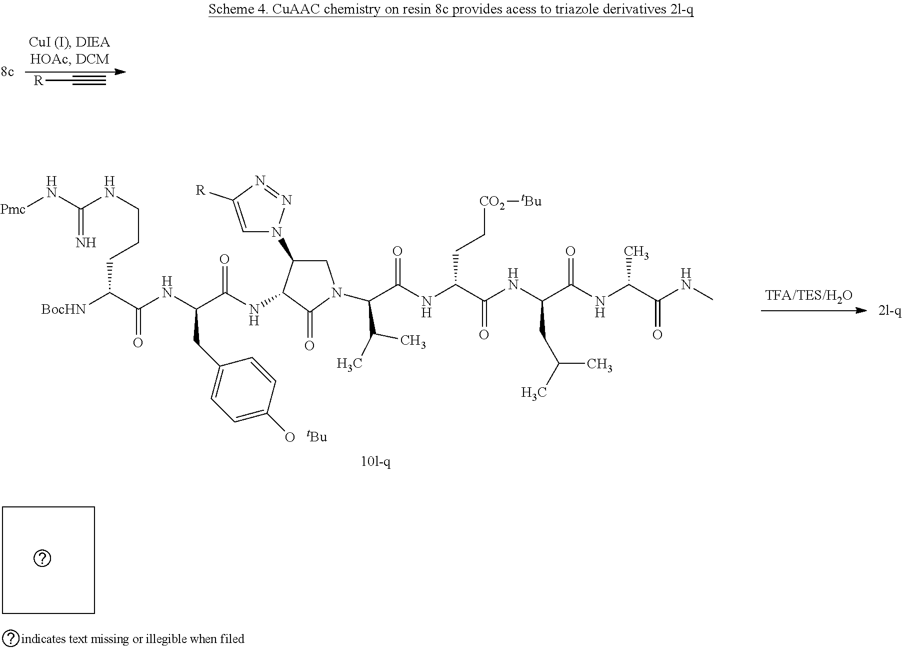

TABLE 2
Retention times, purities, yields, and mass spectrometric data for peptides 2c-q
2c-q

| | 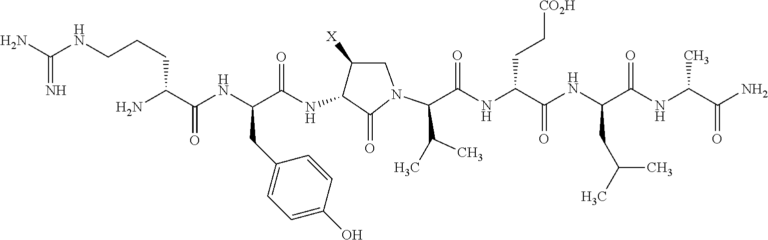 | | | | | | | |
|---|---|---|---|---|---|---|---|---|
| | | RT (min) | | crude | final | yield % | HRMS [M + 1] | |
| 2 | —X | MeOH | MeCN | purity % | purity % | (>95 % in MeOH | m/z (calcd) | m/z (obsd) |
| c | —N₃ | 8.3ª | 5.8ª | 80 | >99 | 22 | 873.4690 | 873.4680 |
| d | —SCN | 8.3ª | 5.8ª | 38 | >96 | 3 | 889.4349 | 889.4342 |
| e | —SMe | 8.7ª | 5.9ª | 46 | >99 | 10 | 878.4553 | 878.4559 |
| f | —OPhth | 8.8c | 7.3^{d} | 48 | >96 | 11 | 993.4789 | 993.4786 |
| g | —ONH₂ | 6.6ª | 5.2ª | 38 | >97 | 5 | 863.4661 | 863.4687 |
| h | —NH₂ | 5.8ª | 1.0^{b} | 70 | >99 | 18 | 847.4785 | 847.4780 |
| i | —NH(C═O)Me | 7.5ª | 5.9ª | 41 | >99 | 9 | 890.4850 | 890.4867 |
| j | —NH(C═O)NH₂ | 7.3ª | 5.1ª | 57 | >99 | 8 | 890.4843 | 890.4841 |
| k | —NH(C═N)NH₂ | 5.9ª | 4.6^{d} | 62 | >98 | 9 | 889.5003 | 889.5006 |
| l | -4-(Ph)triazolyl | 9.6ª | 6.5ª | 74 | >99 | 17 | 975.5159 | 975.5147 |
| m | -4-(p-MeC₆H₄)-triazolyl | 9.5^{e} | 6.5^{f} | 71 | >97 | 16 | 989.5316 | 989.5307 |
| n | -4-(m-H₂NC₆H₄)triazolyl | 7.9^{d} | 6.1^{d} | 69 | >96 | 12 | 990.5268 | 990.5259 |
| o | -4-(H₂N(H₃C)₂C)-triazolyl | 6.3^{d} | 4.9^{d} | 73 | >98 | 13 | 956.5425 | 956.5408 |
| p | -4-(H₂NH₂C)-triazolyl | 7.9^{d} | 6.1^{d} | 53 | >97 | 8 | 928.5039 | 928.5029 |
| q | -4-(HOH₂C)-triazolyl | 7.2^{d} | 5.7^{d} | 64 | >97 | 10 | 929.4930 | 929.4952 |

Isolated purity ascertained by LC-MS analysis using gradients X-Y% MeOH (0.1% FA) or MeCN (0.1% FA) in H₂O (0.1% FA) over 10 min.
^{a}10-90%, ^{b}50-90%, ^{c}30-60%, ^{d}5-60%, ^{e}30-90%, ^{f}20-40%.

### Example 3

### Circular Dichroism Spectra

The impact of the β-substituent on the conformation of (3R,4S)-β-substituted-Agl³ peptides 2c-q was examined in water by CD spectroscopy and the curve shapes of the spectra were compared with that of [(3R,4S)-Hgl³]-1 (2b). Previously, 2b exhibited negative and positive maximum, that were respectively at 198-207 and 221-227 nm indicative of a B-turn conformation in water, trifluoroethanol (TFE), MeOH and hexafluoroisopropanol (HFIP), with the greatest ellipticity seen in 5% TFE in water. In general, peptides 2 exhibited curve shapes indicative of β-turn conformers with slightly different ellipticities (FIG. 2). Notably, thiocyanate 2d exhibited a similar curve shifted to higher wavelengths at 215 and 230 nm (FIG. 2). On the other hand, no identifiable curve shape was obtained from measuring the CD spectrum of 4-hydroxymethyltriazolyl peptide 2q. The similar curve shapes illustrated in the spectra of peptides 2b-p indicated that changes of the β-substituent had little influence on the peptide conformation.

### Example 4

### Biological Activity of the Compounds

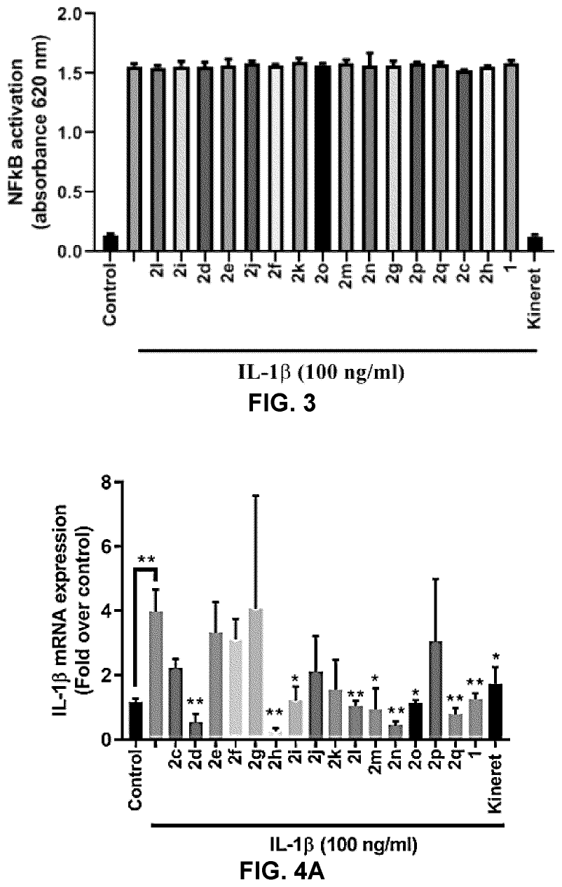

Anti-inflammatory agents that modulate the IL-1R but preserve NF-κB signaling are desired to avoid compromising immune vigilance against invading pathogens. Peptides 2c-q were examined for their effects on the NF-κB pathway using a reported assay. The effects of peptides 1 and 2c-q on the activation of NF-κB signaling by IL-1β was assessed using the QUANTI-blue assay, which quantifies the secretion of alkaline phosphatase, a reporter gene product for NF-κB. Peptides 2c-q all behaved like 1 in the assay and exhibited no effect on IL-1β-induced NF-κB signaling (FIG. 3). On the other hand, Kineret® blocked NF-κB signaling in line with the activity of an orthosteric antagonist.

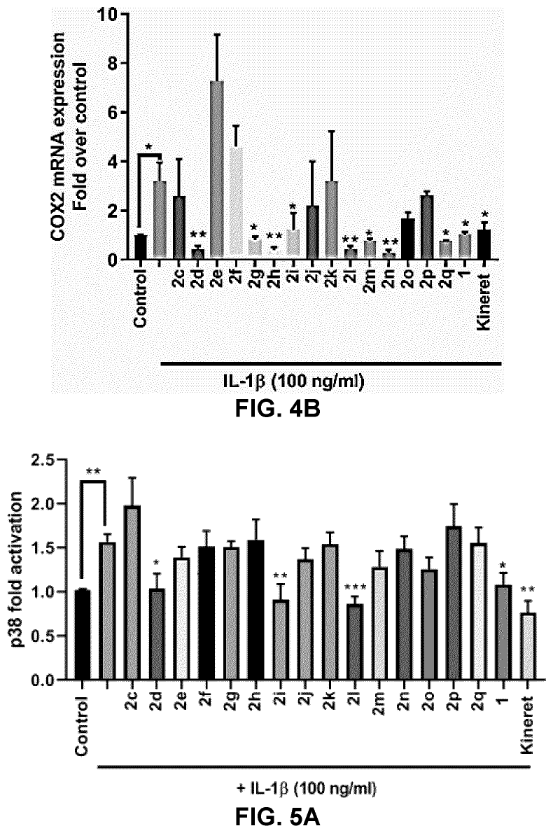

The effects of peptides 1 and 2 on the expression of pro-inflammatory genes for IL-1β and COX-2 were examined in vitro in 264.7 RAW mouse macrophages. Four peptides exhibited statistically (**p<0.01) similar or better activity than peptide 1 and Kineret® in reducing pro-inflammatory gene expression: e.g., thiocyanate 2d, amine 2h, 4-phenyltriazole 2l and 4-m-aminophenyltriazole 2n (FIG. 4). Another three (2o, 2m and 2q) had strong inhibitory effects with lower statistical relevance ('p<0.05) while 2g and 2i blocked expression of COX-2 (p<0.05) without effecting IL-1β expression.

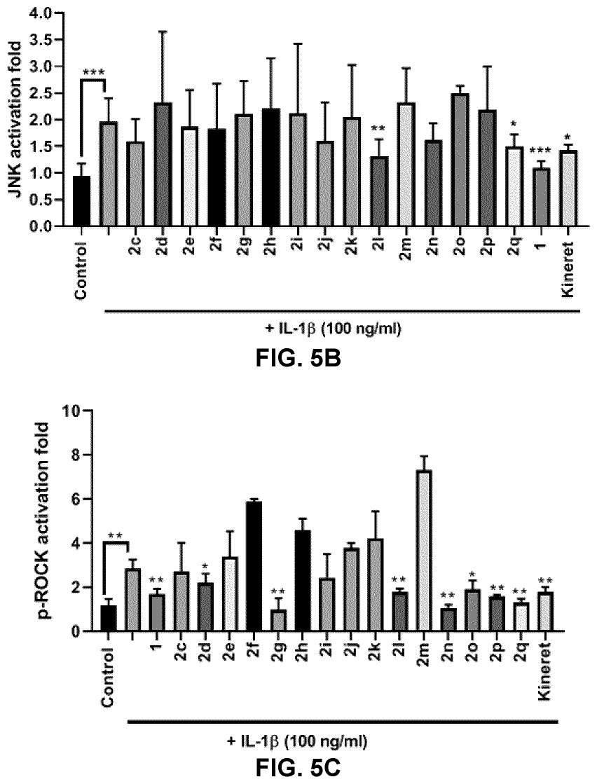

The modulatory effects of peptides 1 and 2 were also determined on IL-1β-induced kinase phosphorylation (FIG. 5). Thiocyanate 2d, amine 2i, and phenyltriazol 2l were the only peptides inhibiting p38 phosphorylation. Only 2l and 2q inhibited activation of JNK. Many β-substituted Agl analogs 2 exhibited inhibitory activity on ROCK2 phosphorylation (e.g., 2g and 2l). All triazolyl analogs 2l-q, except for 4-(p-tolyl)triazole 2m, strongly inhibited ROCK2 phosphorylation activity.

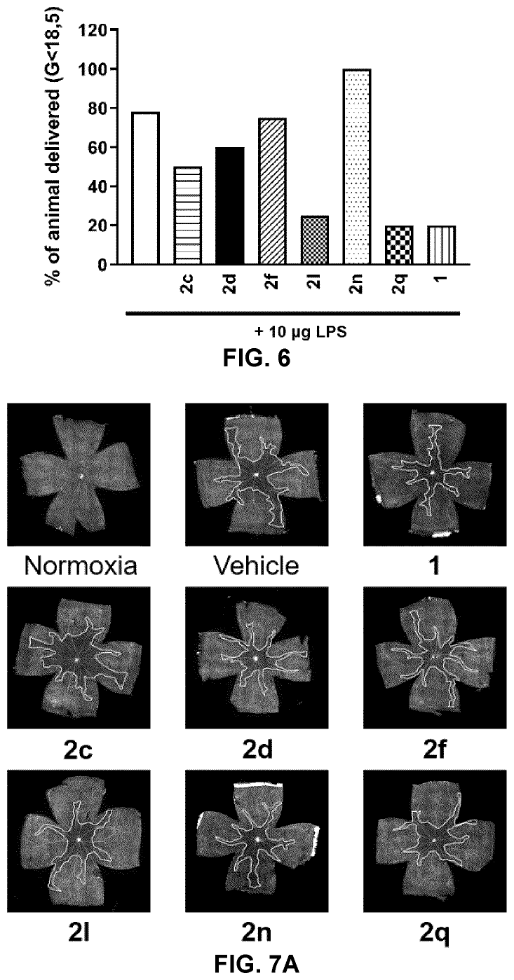

From the results of the in vitro screens, a subset of six (3R,4S)-β-substituted-Agl³ peptides (e.g., 2c, 2d, 2f, 2l, 2n, 2q) were selected for examination in vivo in a CD-1 mouse model of preterm birth (PTB), and a Sprague Dawley rat model of oxygen-induced retinopathy (OIR). In the PTB model, timed-pregnant CD-1 dams were pre-treated with peptides 1 or 2, or PBS vehicle, and then injected with LPS on day 16.5 of gestation (G16.5). A bacterial cell wall component that contains PAMPs, LPS is known to reliably induce labor via pro-inflammatory pathways implicating IL-1 (Hirsch and Wang 2005). The CD-1 mice have a gestation of 19.2 days (Goupil et al. 2010). Dams that delivered at least one pup before G18.5 were considered to have given birth prematurely. In the absence of peptide, LPS alone induced premature labor in ˜80% of treated mice (FIG. 6). Peptide 1 and triazoles 2 l and 2q, all reduced the PTB rate to ˜20%. Azide 2c and thiocyanate 2d, both exhibited modest effects reducing the PTB rate to 40-50%. Neither N-oxyphthalimide 2f nor 4-m-aminophenyltriazole 2n exhibited effects on PTB.

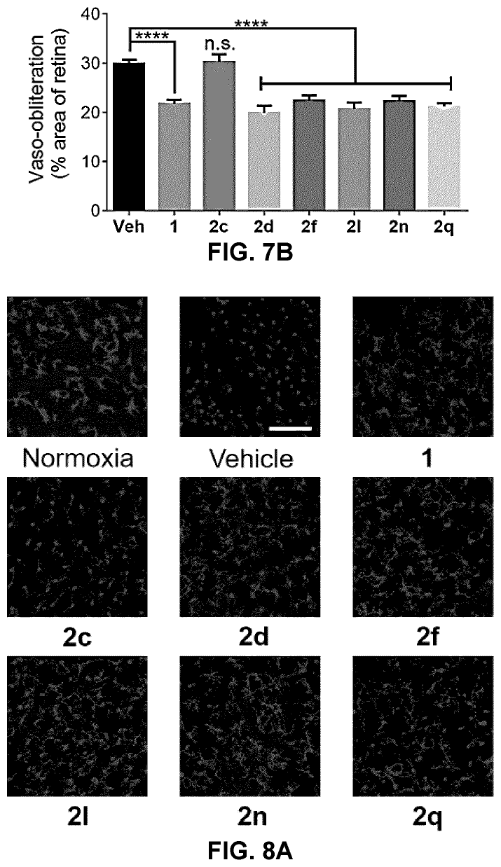

The efficacy of peptides 1 and 2 were compared in the well-established OIR model (a model of retinopathies such as diabetic retinopathy and retinopathy of prematurity) in Sprague Dawley rats as previously described (Geranurimi et al. 2019). After birth, rat pups were exposed to 80% oxygen from days 5 to 10, which usually resulted in ˜30% vaso-obliteration of the retinal capillaries that extend radially from the optic nerve (vehicle, FIGS. 7A and 7B). Peptide 1 diminished the extent of vaso-obliteration from ˜30% to ˜20% (p<0.0001). Five among the six peptides examined, thiocyanate 2d, N-oxyphthalimide 2f, 4-phenyltriazole 2l, 4-m-aminophenyltriazole 2n, and 4-hydroxymethyltriazole 2q, all exhibited efficacy in the OIR model and reduced vaso-obliteration from ˜30% to ˜20% (p<0.0001). Thiocyanate 2d and 4-phenyltriazole 2l exhibited tendencies to have a stronger protective effect than peptide 1, whereas [(3R, 4S)-4-(N₃)Agl³]-1 (2c) had no detectable protective effect against vaso-obliteration.

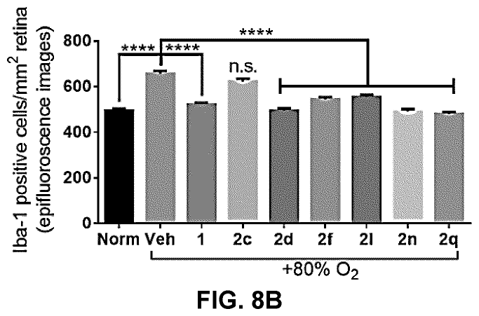

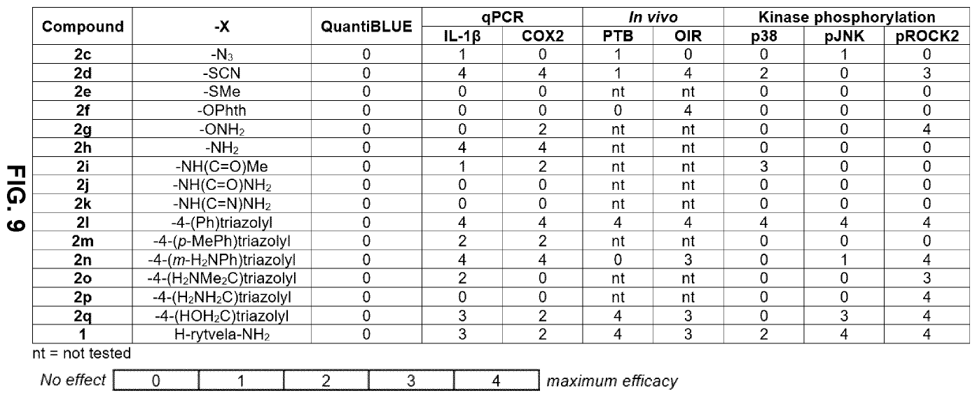

In the context of OIR, microglia have been previously shown to be mediators of vaso-obliteration (Rivera et al. 2013). The ramified and branched morphology of inactive microglia has also been observed to change to an amoeboid state with retracted limbs upon microglial activation (Donat et al. 2017). Microglial activation and density were thus ascertained by histochemical staining for the Iba-1 marker. The active amoeboid state was observed in the retina of animals kept under hypoxia and treated with vehicle or azide 2c (FIGS. 8A and 8B). Pups raised in normoxia exhibited ramified and branched microglia in their retina. Furthermore, pups presented retina with similarly ramified and branched microglia after exposure to hypoxia and treatment respectively with peptides 1 and 2 (2d, 2f, 2l, 2n and 2q), which exhibited diminishing effects on vaso-obliteration. In summary, five of the six tested (3R,4S)-β-substituted-Agl³ peptides acted like peptide 1 and exhibited protection against vaso-obliteration in the hyperoxic phase of OIR, due in part to attenuation of microglial activation.

The results in the various models are summarized in Table 3 (FIG. 9).

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Agalave, S. G., Maujan, S. R. and Pore, V. S. (2011) Click chemistry: 1,2,3-triazoles as pharmacophores. Chem. Asian J., 6, 2696-718. doi: 10.1002/asia.201100432.
Amano, M., Nakayama, M. and Kaibuchi, K. (2010) Rho-kinase/ROCK: a key regulator of the cytoskeleton and cell polarity. Cytoskeleton, 67, 545-54. doi: 10.1002/cm.20472.
Beaudry-Richard, A., Nadeau-Vallee, M., Prairie, É., Maurice, N., Heckel, É., Nezhady, M., Pundir, S., Madaan, A., Boudreault, A., Hou, X., Quiniou, C., Sierra, E.M., Beaulac. A., Lodygensky, G., Robertson, S.A., Keelan, J., Adams Waldorf, K.M., Olson, D.M., Rivera, J.C., Lubell, W.D., Joyal, J.S., Bouchard, J.F. and Chemtob, S. (2018) Antenatal IL-1-dependent inflammation persists postnatally and causes retinal and sub-retinal vasculopathy in progeny. Sci. Rep. 8, 11875. doi: 10.1038/s41598-018-30087-4.
Choi, K.S., Scorpio, D.G. and Dumler, J.S. (2004) Anaplasma phagocytophilum ligation to toll-like receptor (TLR) 2, but not to TLR⁴, activates macrophages for nuclear factor- B nuclear translocation. J. Infect. Dis. 189, 1921-25. doi: 10.1086/386284.
Dianati, V., Shamloo, A., Kwiatkowska, A., Desjardins, R., Soldera, A., Day, R. and Dory, Y.L. (2017) Rational Design of a Highly Potent and Selective Peptide Inhibitor of PACE4 by Salt Bridge Interaction with D160 at Position P3. ChemMedChem 12(15), 1169-1172. doi: 10.1002/cmdc.201700300.
Dinarello, C. A. (1997) Interleukin-1. Cytokine Growth Factor Rev. 8, 253-65. doi: 10.1016/S1359-6101(97)₀₀₀₂₃-3.
Donat, C.K., Scott, G., Gentleman, S.M. and Sastre, M. (2017) Microglial activation in traumatic brain injury. Front Aging Neurosci. 9, 208. doi: 10.3389/fnagi.2017.00208.
Elovitz, M.A., Wang, Z., Chien, E.K., Rychlik, D.F. and Phillippe, M. (2003) A new model for inflammation-induced preterm birth: the role of platelet-activating factor and Toll-like receptor-4. Am. J. Pathol. 163, 2103-11. doi: 10.1016/S0002-9440(10)₆₃₅₆₇-5.
Freidinger, R. M. (2003) Design and synthesis of novel bioactive peptides and peptidomimetics. J. Med. Chem. 46, 5553-66. doi: 10.1021/jm030484k.
Freidinger, R.M., Veber, D.F., Perlow, D.S., Brooks, J.R. and Saperstein R. (1980) Bioactive conformation of luteinizing hormone-releasing hormone: evidence from a conformationally constrained analog. Science 210, 656-58. doi: 10.1126/science.7001627.
Gabay, C., Lamacchia, C. and Palmer, G. (2010) IL-1 pathways in inflammation and human diseases. Nat Rev Rheumatol. 6, 232-41. doi: 10.1038/nrrheum.2010.4.
Geranurimi, A., Cheng, C.W.H., Quiniou, C., Zhu, T., Hou, X., Rivera, J.C., St-Cyr, D.J., Beauregard, K., Bernard-Gauthier, V., Chemtob, S. and Lubell, W.D. (2019) Probing Anti-inflammatory Properties Independent of NF-□B Through Conformational Constraint of Peptide-Based Interleukin-1 Front. Receptor Biased Ligands. Front Chem. 7, 23. doi: 10.3389/fchem.2019.00023.
Geranurimi, A. and Lubell, W. D. (2018) Diversity-oriented syntheses of β-substituted α-amino γ-lactam peptide mimics with constrained backbone and side chain residues. Org. Lett. 20, 6126-29. doi: 10.1021/acs.orglett.8b02575.
Geranurimi, A .; Lubell, W. D. (2018) “Stereoselective synthesis of a □-methylthio α-amino γ-lactam dipeptide, a S-methyl-Cys-Val mimic.” In Proceedings of the 35th European Peptide Symposium Eds. P. B. Timmons, Ch. M. Hewage and M. Lebl (Dublin City University, Ireland), 18-20.
Goupil, E., Tassy, D., Bourguet, C., Quiniou, C., Wisehart, V., Petrin, D., Le Gouill, C., Devost, D., Zingg, H.H., Bouvier, M., Saragovi, H.U., Chemtob, S., Lubell, W.D., Claing, A., Hébert, T.E. and Laporte, S.A. (2010) A novel biased allosteric compound inhibitor of parturition selectively impedes the prostaglandin F2alpha-mediated Rho/ROCK signaling pathway. J. Biol. Chem. 285, 25624-36. doi: 10.1074/jbc.M110.115196.
Gulea, M., Hammerschmidt, F., Marchand, P., Masson, S., Pisljagic, V. and Wuggenig, F. (2003) Synthesis of chiral, nonracemic a-sulfanylphosphonates and derivatives. Tetrahedron: Asymmetry 14, 1829-36. Doi: 10.1016/S0957-4166(03)₀₀₃₁₆-1.
Guthrie, Q.A.E. and Proulx, C. (2018) Oxime Ligation via in situ Oxidation of N-Phenylglycinyl Peptides. Org. Lett. 20, 2564-67. doi: 10.1021/acs.orglett.8b00713.
Hirsch, E. and Wang, H. (2005) The molecular pathophysiology of bacterially induced preterm labor: insights from the murine model. J. Soc. Gynecol. Investig. 12, 145-55. doi: 10.1016/j.jsgi.2005.01.007.
Ilievski, V., Lu, S.J. and Hirsch, E. (2007) Activation of toll-like receptors 2 or 3 and preterm delivery in the mouse. Reprod. Sci. 14, 315-20. doi: 10.1177/1933719107302959.
Jamieson, A. G., Boutard, N., Beauregard, K., Bodas, M. S., Ong, H., Quiniou, C., Chemtob, S. and Lubell, W. D. (2009) Positional scanning for peptide secondary structure by systematic solid-phase synthesis of amino lactam peptides. J. Am. Chem. Soc. 131, 7917-27. doi: 10.1021/ja9010628.
Kaneko, N., Kurata, M., Yamamoto, T., Morikawa, S. and Masumoto, J. (2019) The role of interleukin-1 in general pathology. Inflamm. Regen. 39: 12. doi: 10.1186/s41232-019-0101-5.
Kim, Y.M., Romero, R., Chaiworapongsa, T., Kim, G.J., Kim, M.R., Kuivaniemi, H., Tromp, G., Espinoza, J., Bujold, E., Abrahams, V.M. and Mor, G. (2004) ‘Toll-like receptor-2 and-4 in the chorioamniotic membranes in spontaneous labor at term and in preterm parturition that are associated with chorioamnionitis. Am. J. Obstet. Gynecol. 191, 1346-55. doi: 10.1016/j.ajog.2004.07.009.
Krumm, B., Xiang, Y. and Deng, J. (2014) Structural biology of the IL-1 superfamily: Key cytokines in the regulation of immune and inflammatory responses. Protein Sci. 23, 526-38. doi: 10.1002/pro.2441.
Lubell, W. D., Blankenship, J. W., Fridkin, G., and Kaul, R. (2005). “Peptides,” in Science of Synthesis, ed. S. M. Weinreb (Stuttgart: Thieme), 713-809.
Mantovani, A., Dinarello, C.A., Molgora, M. and Garlanda, C. (2019) Interleukin-1 and Related Cytokines in the Regulation of Inflammation and Immunity. Immunity. 50(4), 778-795. doi: 10.1016/j.immuni.2019.03.012
Melendez, R. E. and Lubell, W.D. (2003) Synthesis and reactivity of cyclic sulfamidites and sulfamidates. Tetrahedron 15, 2581-616. doi: 10.1002/chin.200327214
Nadeau-Vallee, M., Quiniou, C., Palacios, J., Hou, X., Erfani, A., Madaan, A., Sanchez, M., Leimert, K., Boudreault, A., Duhamel, F., Rivera, J.C., Zhu, T., Noueihed, B., Robertson, S.A., Ni,
X., Olson, D.M., Lubell, W., Girard, S. and Chemtob, S. (2015) Novel Noncompetitive IL-1 Receptor-Biased Ligand Prevents Infection- and Inflammation-Induced Preterm Birth. J. Immunol. 195, 3402-15. doi: 10.4049/jimmunol. 1500758.
Opal, S. M., Fisher, C. J., Dhainaut, J. F., Vincent, J. L., Brase, R., Lowry, S. F., et al. (1997). Confirmatory interleukin-1 receptor antagonist trial in severe sepsis: a phase III, randomized, double-blind, placebo-controlled, multicenter trial. the interleukin-1 receptor antagonist sepsis investigator group. Crit. Care Med. 25, 1115-1124. doi: 10.1097/00003246-199707000-00010
Pandey, A.K., Naduthambi, D., Thomas, K.M. and Zondlo, N.J. (2013) Proline editing: a general and practical approach to the synthesis of functionally and structurally diverse peptides. Analysis of steric versus stereoelectronic effects of 4-substituted prolines on conformation within peptides. J. Am. Chem. Soc. 135, 4333-63. doi: 10.1021/ja3109664.
Pirianov, G., Macintyre, D. A., Lee, Y., Waddington, S. N., Terzidou, V., and Mehmet, H., et al. (2015). Specific inhibition of C-Jun N-terminal kinase delays preterm labour and reduces mortality. Reproduction 150, 269-277. doi: 10.1530/REP-15-0258
Quiniou, C., Sapieha, P., Lahaie, I., Hou, X., Brault, S., Beauchamp, M., et al. (2008). Development of a novel noncompetitive antagonist of IL-1 receptor. J. Immunol. 180, 6977-6987. doi: 10.4049/jimmunol. 180.10.6977
Rivera, J.C., Sapieha, P., Honore, J.C., Hamel, D., Quiniou, C. and Chemtob, S. (2010) A Novel Modulator of the IL-1 Receptor Prevents Development of Oxygen-Induced Retinopathy. Invest. Ophthalmol. Vis. Sci. 51, 3336-36.
Rivera, J. C., Sitaras, N., Noueihed, B., Hamel, D., Madaan, A., Zhou, T., et al. (2013). Microglia and interleukin-1b in ischemic retinopathy elicit microvascular degeneration through neuronal semaphorin-3A. Arterioscler. Thromb. Vasc. Biol. 33, 1881-1891. doi: 10.1161/ATVBAHA. 113.301331
Rivera, J.C., Holm, M., Austeng, D., Morken, T.S., Zhou, T.E., Beaudry-Richard, A., Sierra, E.M., Dammann, O. and Chemtob, S. (2017) Retinopathy of prematurity: inflammation, choroidal degeneration, and novel promising therapeutic strategies. J. Neuroinflammation. 14, 165. doi: 10.1186/s12974-017-0943-1.
Roerink, M.E., van der Schaaf, M.E., Dinarello, C.A., Knoop, H. and van der Meer, J.W. (2017) Interleukin-1 as a mediator of fatigue in disease: a narrative review. J. Neuroinflammation. 14, 16. doi: 10.1186/s12974-017-0796-7.
Romero, R., Brody, D. T., Oyarzun, E., Mazor, M., Wu, Y. K., Hobbins, J. C., et al. (1989). Infection and labor. III. Interleukin-1: a signal for the onset of parturition. Am. J. Obstetr. Gynecol. 160 (Pt 1), 1117-1123. doi: 10.1016/0002-9378(89)₉₀₁₇₂-5.
Ronga, L., Jamieson, A. G., Beauregard, K., Quiniou, C., Chemtob, S., and Lubell, W. D. (2010). Insertion of multiple alpha-amino gamma-lactam (Agl) residues into a peptide sequence by solid-phase synthesis on synphase lanterns. Biopolymers 94, 183-191. doi: 10.1002/bip.21288.
Roy, P.K., Rashid, F., Bragg, J. and Ibdah, J.A. (2008) Role of the JNK signal transduction pathway in inflammatory bowel disease. World J. Gastroenterol. 14, 200-2. doi: 10.3748/wjg. 14.200.
Shao, C., Wang, X., Zhang, Q., Luo, S., Zhao, J. and Hu, Y. (2011) Acid-base jointly promoted copper (I)-catalyzed azide-alkyne cycloaddition. J. Org. Chem. 76, 6832-36. doi: 10.1021/jo200869a.
St-Cyr, D. J., Jamieson, A. G., and Lubell, W. D. (2010). Alpha-aminobeta- hydroxy-gamma-lactam for constraining peptide Ser and Thr residue conformation. Org. Lett. 12, 1652-1655. doi: 10.1021/ol1000582
St-Cyr, D. J., García-Ramos, Y., Doan, N. D., and Lubell, W. D. (2017). “Aminolactam, N-aminoimidazolone, and N-aminoimdazolidinone peptide mimics,” in Peptidomimetics I, ed. W. D. Lubell (Cham: Springer), 125-175.
Tosato, G. and Jones, K.D. (1990) Interleukin-1 induces interleukin-6 production in peripheral blood monocytes. Blood 75, 1305-10.
Tremblay, S., Miloudi, K., Chaychi, S., Favret, S., Binet, F., Polosa, A., Lachapelle, P., Chemtob, S. and Sapieha, P. (2013) Systemic inflammation perturbs developmental retinal angiogenesis and neuroretinal function. Invest. Ophthalmol. Vis. Sci. 54, 8125-39. doi: 10.1167/iovs. 13-12496.
Villadsen, K., Martos-Maldonado, M.C., Jensen, K.J. and Thygesen, M.B. (2017) Chemoselective reactions for the synthesis of glycoconjugates from unprotected carbohydrates. ChemBioChem 18, 574-612. doi: 10.1002/cbic.201600582.
Wertheim, E. (1931) Derivatives of Dulcin. J. Am. Chem. Soc. 53, 200-03. doi: 10.1021/ja01352a026.
Xu, W.Q. and Wang, Y.S. (2016) The role of Toll-like receptors in retinal ischemic diseases. Int. J. Ophthalmol. 9, 1343-51. doi: 10.18240/ijo.2016.09.19.
Yamaguchi, M., Nakao, S., Arita, R., Kaizu, Y., Arima, M., Zhou, Y., et al. (2016). Vascular normalization by ROCK inhibitor: therapeutic potential of Ripasudil (K-115) eye drop in retinal angiogenesis and hypoxia. Invest. Ophthamol. Vis. Sci. 57, 2264-2276. doi: 10.1167/iovs. 15-17411
Zhou, T.E., Rivera, J.C., Bhosle, V.K., Lahaie, I., Shao, Z., Tahiri, H., Zhu, T., Polosa, A., Dorfman, A., Beaudry-Richard, A., Costantino, S., Lodygensky, G.A., Lachapelle, P., Chemtob, S. (2016) Choroidal involution is associated with a progressive degeneration of the outer retinal function in a model of retinopathy of prematurity: early role for IL-1β. Am. J. Pathol. 186, 3100-16. doi: 10.1016/j.ajpath.2016.08.004.

1. A compound of formula (I): 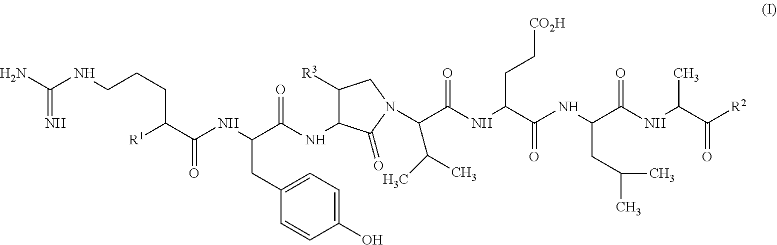
wherein:
R¹ is H or NR⁴R⁵, wherein R⁴ and R⁵ are each independently H or R^{A}p13 CO—, wherein R^{A} is a hydrophobic moiety or an aryl group;
R² is H, OH, C₁-6 alkyl, O-C₁-6 alkyl or NR⁶R⁷, wherein R⁶ and R⁷ are each independently H, OH, or C₁-6 alkyl;
R³ is S—C═N, NR⁶R⁷, optionally substituted phenyl; optionally substituted 3- to 7-membered saturated or partially unsaturated carbocyclic ring; optionally substituted 5- or 6-membered monocyclic heteroaryl ring having 1-3 heteroatoms independently selected from nitrogen, oxygen, and sulfur; or optionally substituted 4- to 7-membered saturated or partially unsaturated heterocyclic ring having 1-3 heteroatoms independently selected from nitrogen, oxygen, and sulfur;
or a pharmaceutically acceptable salt thereof.

2. The compound or pharmaceutically acceptable salt thereof of claim 1, wherein R⁴ and/or R⁵ are H.

3. The compound or pharmaceutically acceptable salt thereof of claim 1, wherein R² is NR⁶R⁷.

4. The compound or pharmaceutically acceptable salt thereof of claim 3, wherein R⁶ and/or R⁷ are H.

5. The compound or pharmaceutically acceptable salt thereof of claim 1, wherein R³ is S—C═N.

6. The compound or pharmaceutically acceptable salt thereof of claim 1, wherein R³ is NR⁶R⁷

7. The compound or pharmaceutically acceptable salt thereof of claim 6, wherein R⁶ and/or R⁷ are H.

8. The compound or pharmaceutically acceptable salt thereof of claim 1, wherein R³ is optionally substituted 5- or 6-membered monocyclic heteroaryl ring having 1-3 heteroatoms independently selected from nitrogen, oxygen, and sulfur.

9. The compound or pharmaceutically acceptable salt thereof of claim 8, wherein R³ is optionally substituted 5-membered monocyclic heteroaryl ring having 1-3 nitrogen atoms.

10. The compound or pharmaceutically acceptable salt thereof of claim 9, wherein R³ is 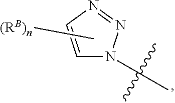
wherein
each R^{B} is independently hydroxy, halogen atom, alkyl, haloalkyl, alkoxy, cyano, amino, alkylamino, nitro, —N(haloalkyl)₂, aryl, heteroaryl, arylalkyl, arylalkyloxy, or —C(O)aryl;
the alkyl, aryl, arylalkyl, heteroaryl, and alkylaryl in R^{B} being optionally substituted with one or more: hydroxy, halogen atom, alkyl, haloalkyl, alkoxy, cyano, amino, alkylamino, nitro, —N(haloalkyl)₂, aryl, arylalkyl, arylalkyloxy, or —C(O)aryl; and
n is 0, 1 or 2.

11. The compound or pharmaceutically acceptable salt thereof of claim 10, wherein R³ is

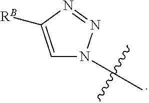

12. The compound or pharmaceutically acceptable salt thereof of claim 10, wherein R³ is: 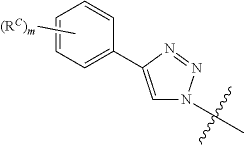
wherein
each R^{C} is independently hydroxy, halogen atom, alkyl, haloalkyl, alkoxy, cyano, amino, alkylamino, nitro, or —N(haloalkyl)₂; and
m is 0, 1, 2 or 3.

13. The compound or pharmaceutically acceptable salt thereof of claim 12, wherein m is 0.

14. The compound or pharmaceutically acceptable salt thereof of claim 1, which is a compound of formula 1a: 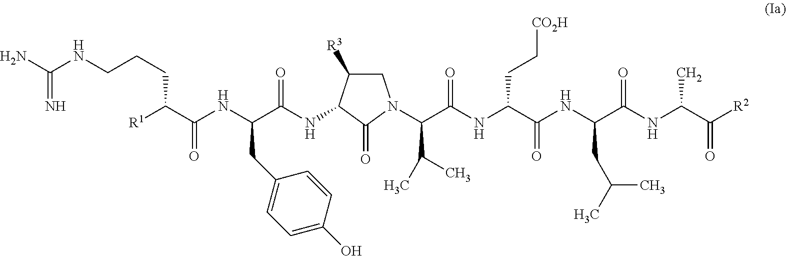
or a pharmaceutically acceptable salt thereof.

15. The compound or pharmaceutically acceptable salt thereof of claim 1, which is a compound of one of the following structures: 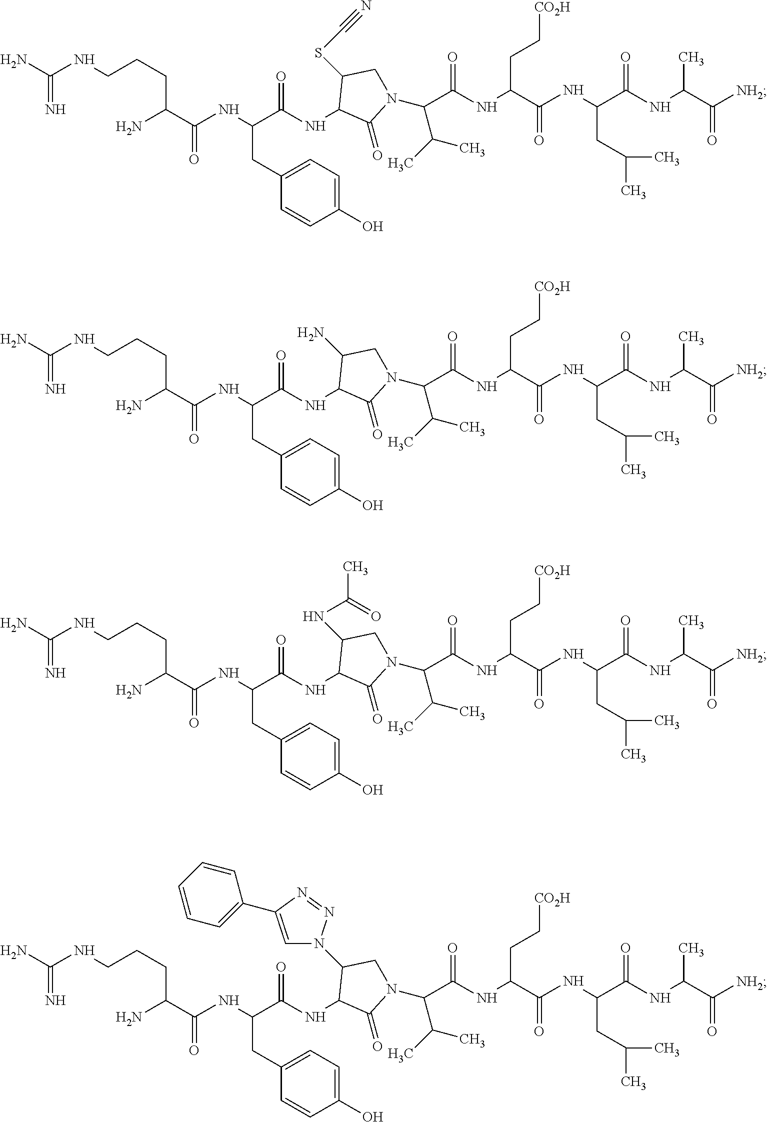 [SMILES: CC(C)CC(NC(=O)C(CCC(=O)O)NC(=O)C(C(C)C)N1CC(SC#N)C(NC(=O)C(Cc2ccc(O)cc2)NC(=O)C(N)CCCNC(=N)N)C1=O)C(=O)NC(C)C(N)=O ; CC(C)CC(NC(=O)C(CCC(=O)O)NC(=O)C(C(C)C)N1CC(N)C(NC(=O)C(Cc2ccc(O)cc2)NC(=O)C(N)CCCNC(=N)N)C1=O)C(=O)NC(C)C(N)=O ; CC(=O)NC1CN(C(C(=O)NC(CCC(=O)O)C(=O)NC(CC(C)C)C(=O)NC(C)C(N)=O)C(C)C)C(=O)C1NC(=O)C(Cc1ccc(O)cc1)NC(=O)C(N)CCCNC(=N)N ; CC(C)CC(NC(=O)C(CCC(=O)O)NC(=O)C(C(C)C)N1CC(n2cc(-c3ccccc3)nn2)C(NC(=O)C(Cc2ccc(O)cc2)NC(=O)C(N)CCCNC(=N)N)C1=O)C(=O)NC(C)C(N)=O] 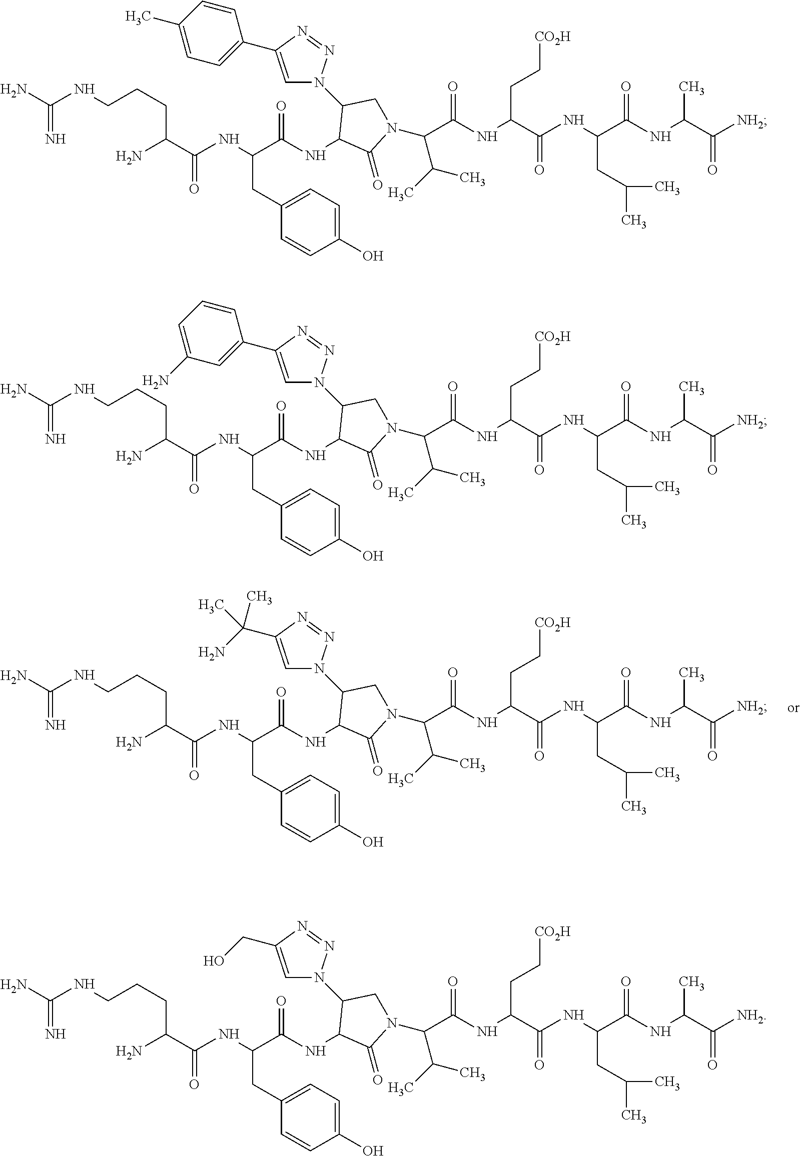
or a pharmaceutically acceptable salt thereof.

16. The compound or pharmaceutically acceptable salt thereof of claim 15, which is a compound of one of the following structures: 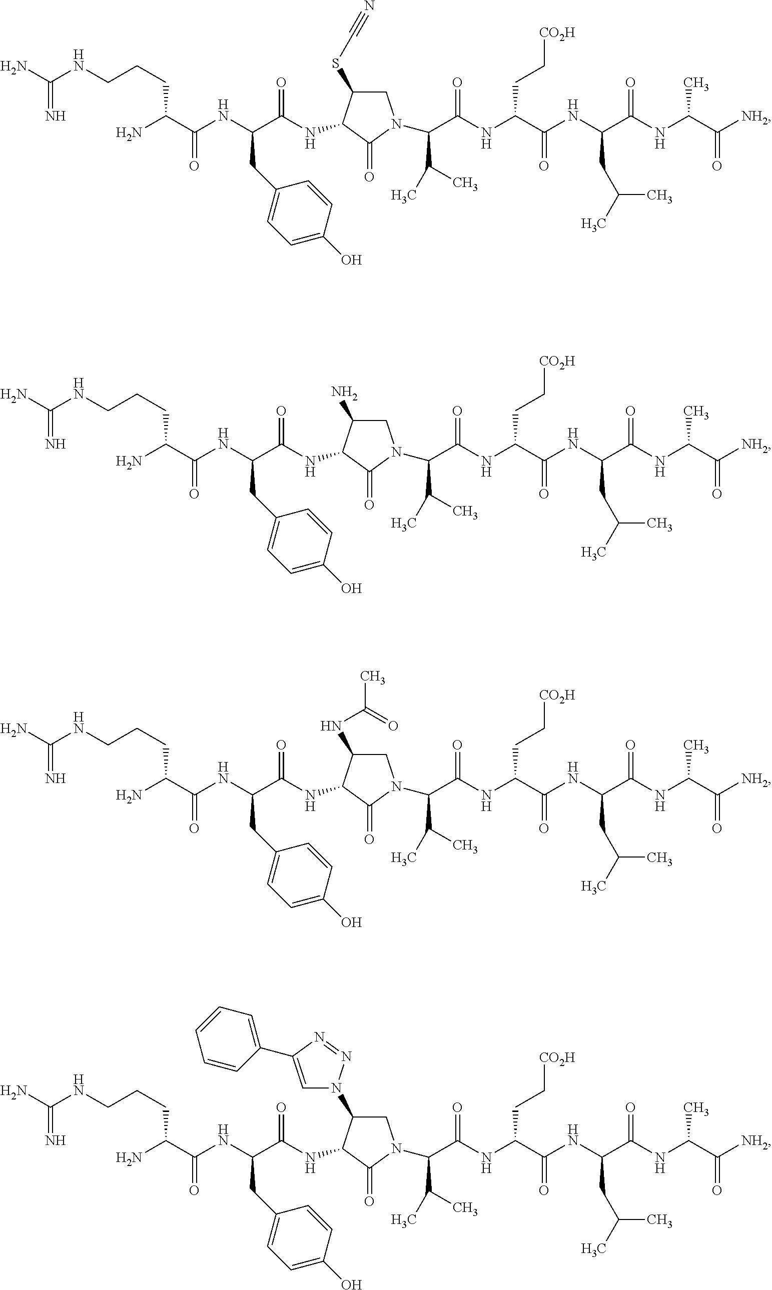 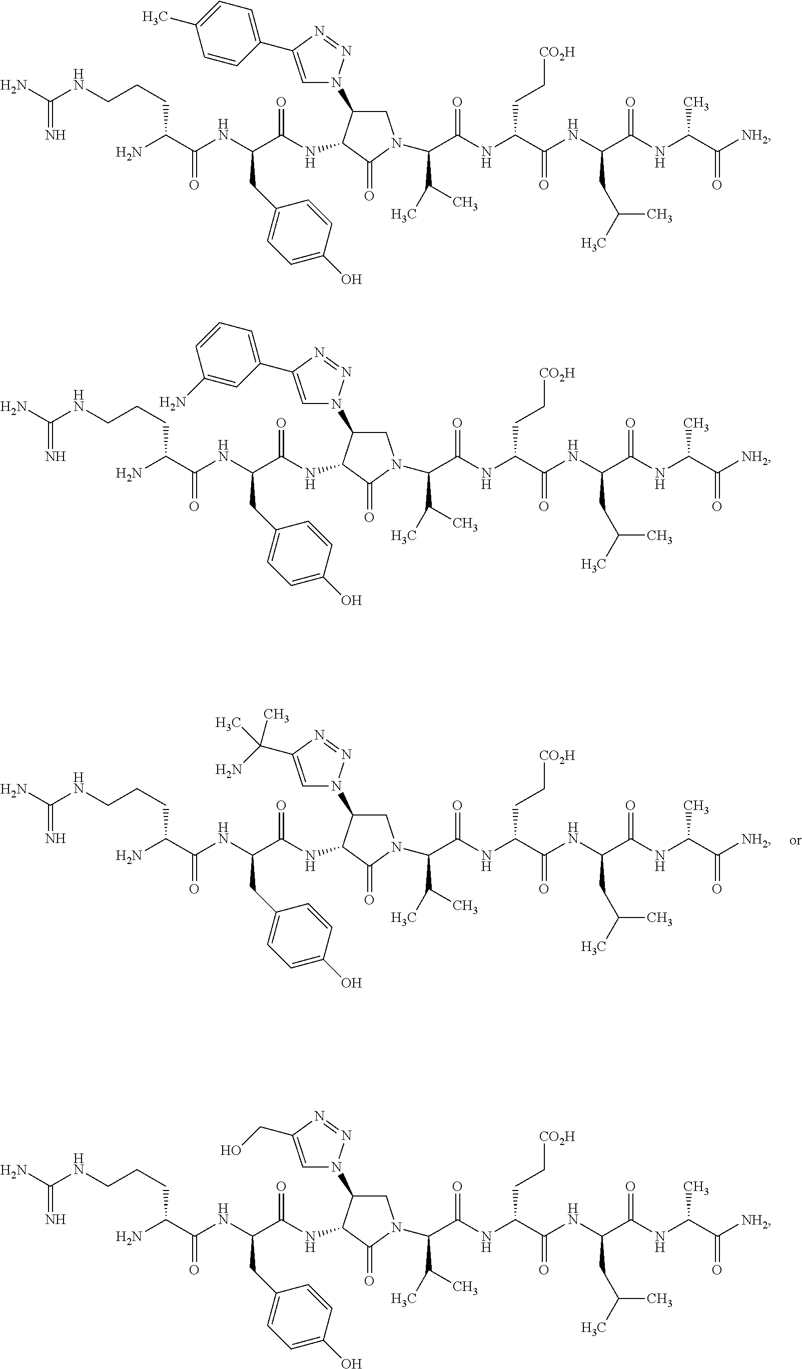
or a pharmaceutically acceptable salt thereof.

17. The compound or pharmaceutically acceptable salt thereof of claim 16, which is 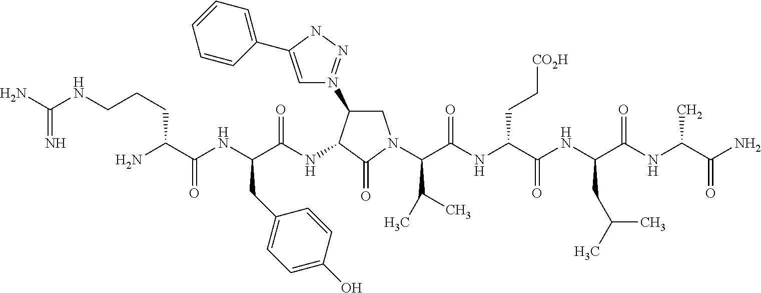
or a pharmaceutically acceptable salt thereof.

18. A pharmaceutical composition comprising the compound or pharmaceutically acceptable salt thereof of claim 1 and a pharmaceutically acceptable excipient.

19-22. (canceled)

23. A method of treating an IL-1 related disease, disorder or condition, the method comprising administering to a subject in need of treatment the compound or pharmaceutically acceptable salt thereof of claim 1.

24. (canceled)

25. The method of claim 23, wherein the IL-1-related disease, disorder or condition is inflammatory joint disease, inflammatory bowel disease, psoriasis, encephalitis, glomerulonephritis, respiratory distress syndrome, Reiter's syndrome, systemic lupus erythematosus, scleroderma, stroke, periventricular leucopenia, meningitis, multiple sclerosis, acute disseminated encephalomyelitis (ADEM), idiopathic inflammatory demyelinating disease, transverse myelitis, Devic's disease, progressive multifocal leukoencephalopathy (PML), Guillain-Barre syndrome, age-related macular degeneration (AMD), retinopathy, chronic inflammatory demyelinating polyneuropathy, anti-MAG neuropathy, sepsis, septic shock, pancreatitis, trauma-induced shock, asthma, allergic rhinitis, cystic fibrosis, acute bronchitis, chronic bronchitis, acute bronchiolitis, chronic bronchiolitis, gout, placental, fetal and/or uterine inflammation, inflammatory condition resulting from an injury, or inflammatory condition resulting from an infection.

26-51. (canceled)


SEQUENCE LISTING (4 sequences)

SEQ ID NO: 1 - DNA, 22 bp, Artificial Sequence
  Synthetic oligonucleotide
agctggagagtgtagatcccaa

SEQ ID NO: 2 - DNA, 20 bp, Artificial Sequence
  Synthetic oligonucleotide
acgggcatgttttctgcttg

SEQ ID NO: 3 - DNA, 20 bp, Artificial Sequence
  Synthetic oligonucleotide
atattggtgacccgtggagc

SEQ ID NO: 4 - DNA, 20 bp, Artificial Sequence
  Synthetic oligonucleotide
gttctccgtaccttcacccc